(19) United States
(12) Patent Application Publication
(10) Pub. No.: US 2023/0333019 A1
(43) Pub. Date: Oct. 19, 2023

(54) TWO-PHOTON FLUORESCENT PROBES FOR SIMULTANEOUSLY DETECTING CALCIUM IONS IN ORGANELLES AND LYSOSOMAL PROTONS

(71) Applicants: Korea University Research and Business Foundation, Seoul (KR); Daejin University Center for Educational Industrial Cooperation, Pocheon-si (KR)

(72) Inventors: Seung Taek HONG, Seoul (KR); Eun-sun KIM, Seoul (KR); Bong Rae CHO, Seoul (KR); Mun Seok KIM, Jinju-si (KR)

(73) Assignees: Korea University Research and Business Foundation, Seoul (KR); Daejin University Center for Educational Industrial Cooperation, Pocheon-si (KR)

(21) Appl. No.: 18/115,303

(22) Filed: Feb. 28, 2023

(30) Foreign Application Priority Data
Feb. 28, 2022 (KR) 10-2022-0025902
Feb. 28, 2022 (KR) 10-2022-0025903
Feb. 28, 2022 (KR) 10-2022-0025904

(51) Int. Cl. G01N 21/64; C07D 263/57

(52) U.S. Cl. CPC G01N 21/6458; G01N 21/6428; C07D 263/57

(57) ABSTRACT

Disclosed are organelles-specific two-photon fluorescent probes, and more particularly, a blue emission two-photon fluorescent probe capable of selectively detecting calcium ions (Ca²⁺) through fluorescence signals by targeting the cytoplasm, mitochondria, and plasma membrane even among the organelles, respectively, and a green emission two-photon fluorescent probe capable of selectively detecting hydrogen ions (H⁺) through fluorescence signals by targeting the lysosome, a method for preparing these two-photon probes, and a method for separately or simultaneously imaging calcium ions in the cytoplasm, mitochondria, or plasma membrane and hydrogen ions in the lysosome using the two two-photon probes.

### TECHNICAL FIELD

The present disclosure relates to a two-photon fluorescent probe (marker) capable of targeting specific organelles and labeling ions to be detected, and more particularly, to a blue emission two-photon fluorescent probe for detecting calcium ions by targeting the cytoplasm, mitochondria, and plasma membrane, respectively, and a green emission two-photon fluorescent probe for detecting hydrogen ions (proton ions) by targeting the lysosome, a manufacturing method of these two-photon probes, and a method for imaging calcium ions in specific organelles and lysosomal hydrogen ions separately or simultaneously using these two-photon probes.

### BACKGROUND

Ca²⁺ is known to perform several important functions in vivo, such as signaling, exocytosis, apoptosis, transcription, muscle contraction, and bone formation. Defects in Ca²⁺ signaling may cause diseases such as neurodegeneration, muscle defects, heart disease, and skin diseases. Calcium (Ca) ions enter the cytoplasm from an extracellular space or internal Ca²⁺ storage such as the endoplasmic reticulum and the mitochondria.

The concentration of free Ca²⁺ is about 1 mM in the extracellular space, whereas the concentrations in the cytoplasm, the endoplasmic reticulum, and the mitochondria are 0.1 to 1 μM, 0.1 to 1 mM, and 0.1 to 20 μM, respectively. The signal is triggered when Ca²⁺ is released from the intracellular storage or introduced into the cell through ion channels.

Fluorescence imaging using fluorescent probes such as Fluo-4, Fura-2, Rhod-2, or Calcium Green-1 is the most common method to study Ca²⁺ biology in the cell. However, these probes require relatively short excitation wavelengths of 350 to 500 nm, which is unsuitable for tissue imaging due to limited penetration depth (<80 μm) and autofluorescence. Two-photon (TP) microscopy (TPM) may enable long-term imaging of live tissue at depths of 500 μm or more to overcome these drawbacks.

The two-photon microscopy has advantages of higher spatial resolution, less photobleaching, and less phototoxicity than one-photon microscopy currently widely used. These advantages are much remarkable when a two-photon fluorescent probe (two-photon marker) with high two-photon absorption and high quantum yield is used together, and it is advantageous for obtaining 3D real-time images of life phenomena occurring in live cells and tissues. However, due to the small number of two-photon fluorescent probes (two-photon markers) which have been currently developed, it was impossible to observe various life phenomena using the two-photon microscopy.

In particular, there is an urgent need to develop an organelle-specific Ca²⁺ TP probe suitable for multicolor imaging for monitoring Ca²⁺ transport from one organelle to the other organelle in live tissue. Accordingly, several TP probes for Ca²⁺ have been developed in the related art, but there is no attempt to develop a Ca²⁺ TP probe suitable for multicolor imaging to realize the purpose. There are also few blue emission TP probes for Ca²⁺.

Therefore, the present inventors synthesized a Ca²⁺ two-photon probe by binding a benzoxazole derivative and a calcium ion receptor suitable for measuring changes in calcium concentration as a result of research to develop a blue light two-photon probe capable of detecting Ca²⁺ in vivo, confirmed that two-photon excitation fluorescence was increased by binding the blue emission two-photon fluorescent probe with the calcium ions in the specific organelles to measure changes in calcium ions specifically in organelles in real time, and then completed the present disclosure.

### SUMMARY

The present disclosure has been made in an effort to provide a blue emission two-photon fluorescent probe compound capable of detecting calcium through fluorescent signals by targeting specific organelles, a preparing method thereof, a two-photon fluorescent probe composition for detecting calcium ions in live cells or tissues containing the compound, and a method for imaging calcium ions in live cells or tissues using the compound.

In order to achieve the above purpose, an embodiment of the present disclosure provides a two-photon fluorescent probe compound represented by any one of Chemical Formulas 1 to 3 below:

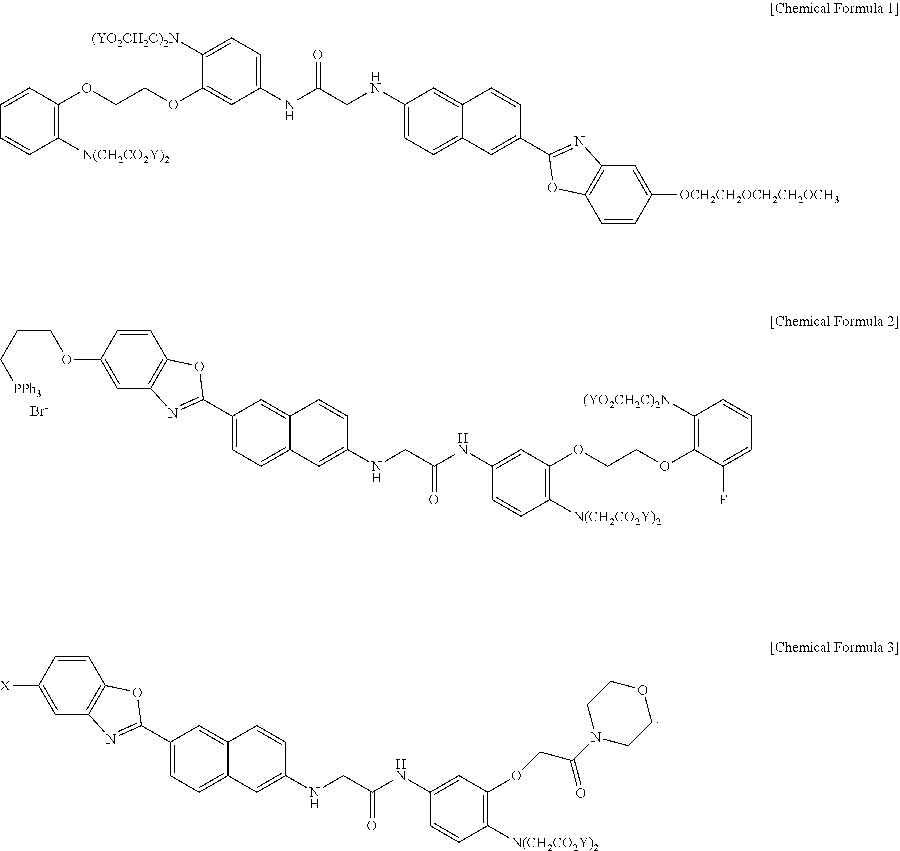

In Chemical Formulas 1 to 3, Y is each independently K, Na, CH₃, or CH₂OC(O)CH₃, and in Chemical Formula 3, X is H, OH, or O(CH₂)₅CH₃.

Another embodiment of the present disclosure provides a two-photon fluorescent probe composition for detecting calcium ions in live cells or tissues including at least one compound selected from the two-photon fluorescent probe compounds represented by any one of Chemical Formulas 1 to 3.

Yet another embodiment of the present disclosure provides a method for imaging calcium ions including injecting at least one compound selected from two-photon fluorescent probe compounds represented by any one of Chemical Formulas 1 to 3 into cells or tissues isolated from a living body; irradiating an excitation source to the cells or tissues isolated from the living body; and observing fluorescence generated from the two-photon fluorescent probe compound by two-photon microscopy.

Still another embodiment of the present disclosure provides a method for preparing a two-photon fluorescent probe compound represented by any one of Chemical Formulas 1 to 3, including reacting a compound represented by Chemical Formula 5 below with a compound represented by Chemical Formula 6 or 7 below:

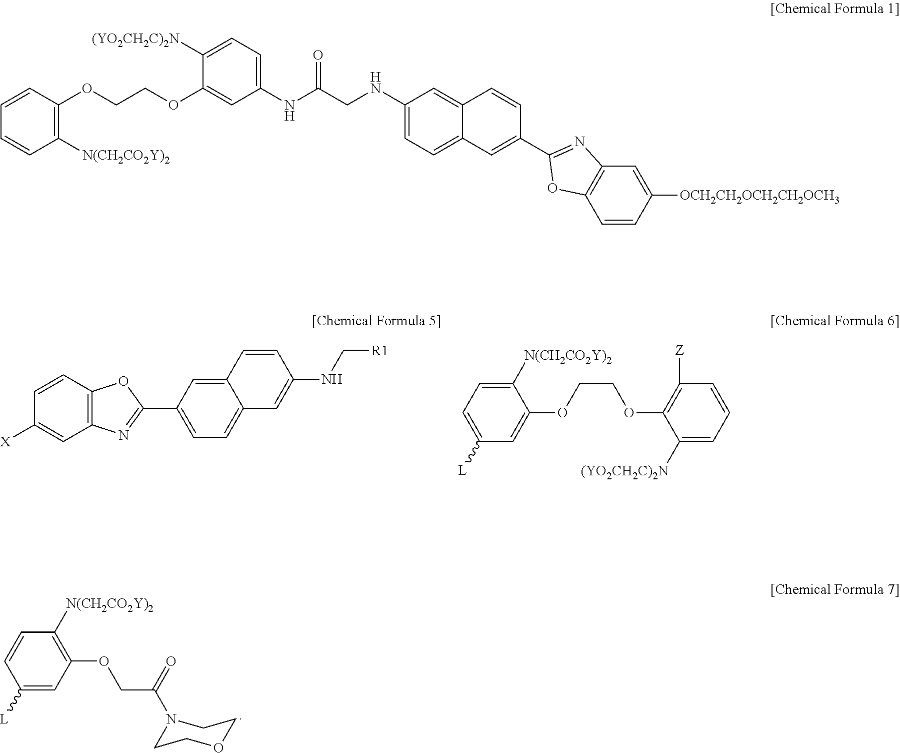

In Chemical Formula 5, 6, or 7, Y is each independently K, Na, CH₃, or CH₂OC(O)CH₃, X is H, OH, O(CH₂CH₂O)₂CH₃, O(CH₂)₃Br, O(CH₂)₃PPh₃⁺Br⁻, or O(CH₂)₅CH₃, R1 is CO₂C(CH₃)₃ or CO₂H, Z is H or F, and L is H or NH₂.

Still yet another embodiment of the present disclosure provides a two-photon fluorescent probe compound represented by Chemical Formula 4 below:

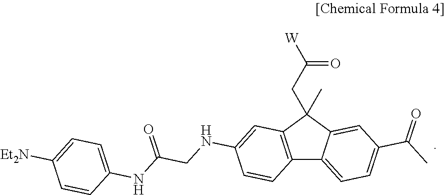

In Chemical Formula 4, W is NH(CH₂)₂N(CH₃)₂ or OCH₃.

Still yet another embodiment of the present disclosure provides a two-photon fluorescent probe composition for detecting hydrogen ions in live cells or tissues, including the two-photon fluorescent probe compound represented by Chemical Formula 4.

Still yet another embodiment of the present disclosure provides a method for imaging hydrogen ions including injecting the two-photon fluorescent probe compound represented by Chemical Formula 4 into cells or tissues isolated from a living body; irradiating an excitation source to the cells or tissues isolated from the living body; and observing fluorescence generated from the two-photon fluorescent probe compound by two-photon microscopy.

Still yet another embodiment of the present disclosure provides a method for preparing a two-photon fluorescent probe compound represented by Chemical Formula 4, including reacting a compound represented by Chemical Formula 8 below with a compound represented by Chemical Formula 9 below:

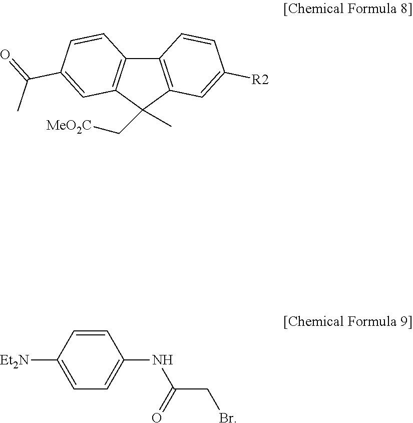

In Chemical Formula 8, R2 is NO₂ or NH₂.

Still yet another embodiment of the present disclosure provides a two-photon fluorescent probe composition for simultaneously detecting calcium ions and hydrogen ions including at least one compound selected from the two-photon fluorescent probe compounds represented by any one of Chemical Formulas 1 to 3 and the compound represented by Chemical Formula 4.

Still yet another embodiment of the present disclosure provides a method for simultaneously imaging calcium ions and hydrogen ions including injecting at least one compound selected from the two-photon fluorescent probe compounds represented by any one of Chemical Formulas 1 to 3 and the two-photon fluorescent probe compound represented by Chemical Formula 4 into cells or tissues isolated from a living body; irradiating an excitation source to the cells or tissues isolated from the living body; and observing fluorescence generated from the two two-photon fluorescent probe compounds by two-photon microscopy.

According to the embodiments of the present disclosure, a blue emission two-photon fluorescent probe for detecting calcium ions is a two-photon fluorescence probe (marker) capable of selectively imaging ions to be detected by targeting specific organelles, that is, the cytoplasm, mitochondria, or plasma membrane, respectively, so that it is possible to provide two-photon probes with low cytotoxicity, high photostability, and pH independence.

In addition, a blue emission two-photon fluorescent probe for detecting calcium ions by targeting the cytoplasm, mitochondria, and plasma membrane, respectively, and a green emission two-photon fluorescent probe for detecting hydrogen ions by targeting the lysosome can simultaneously image calcium ions in the cytoplasm, mitochondria, or plasma membrane and hydrogen ions in the lysosome of live cells and tissues, thereby simultaneously monitoring a distribution of calcium ions and a change in acidity (pH) in vivo.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates results of analyzing the purity of BCa-2ₘᵢₜₒ by mass spectrometry and liquid chromatography: (a) Total ion chromatogram (TIC, top) and UV chromatogram at 254 nm (bottom), (b) Enlargement results of chromatograms of TIC (top panel) and UV (bottom panel) in (a), (c) Mass spectra in ESI positive mode of peaks 1, 2, and 3, and (d) HPLC conditions and integral table of UV peaks.

FIG. 2 is graphs showing absorption spectra of (a) BCa-1, (b) BCa-2ₘᵢₜₒ, (c) BCa−3°ₘₑₘ, and (d) FHEt-1_{lyso} in an aqueous buffer (pH 7.4), and absorbance plots with respect to probe concentrations in (e) 353 nm (BCa-1), (f) 358 nm (BCa-2ₘᵢₜₒ), (g) 350 nm (BCa−3°ₘₑₘ), or (h) 359 nm (FHEt-1_{lyso}) in an aqueous buffer (pH 7.4).

FIG. 3 is fluorescent spectra of (a) BCa-1, (b) BCa-2ₘᵢₜₒ, (c) BCa−3°ₘₑₘ, and (d) FHEt-1_{lyso} in an aqueous buffer (pH 7.2), and plots showing fluorescence intensities with respect to concentrations in (e) BCa-1, (0 BCa-2ₘᵢₜₒ, (g) BCa−3°ₘₑₘ, or (h) FHEt-1 lyso in an aqueous buffer (pH 7.2).

FIG. 4 is a graph showing normalized TPEF spectra at an excitation wavelength of 750 nm for Hela cells labeled with BCa-1-AM, BCa-2 onto-AM, BCa-3ₘₑₘ, and FHEt-1_{lyso}.

FIG. 5 is a graph showing normalized fluorescence spectra for (a) BCa-1, (b) BCa-2ₘᵢₜₒ, (c) BCa-3ₘₑₘ, and (d) FHEt-1_{lyso} in HeLa cells (black curve) or an aqueous buffer (red curve).

FIG. 6 is graphs showing (a) a fluorescence spectrum and (b) a fluorescence titration curve for complexation with Ca ions of BCa-1 in the presence of 0 to 39 μM free Ca²⁺ in CB; (c) a fluorescence spectrum and (b) a fluorescence titration curve for complexation with Ca ions of BCa-2ₘᵢₜₒ in the presence of 0 to 2.5 mM free Ca²⁺ in CB; (e) a fluorescence spectrum and (0 a fluorescence titration curve for complexation with Ca ions of BCa−3°ₘₑₘ in the presence of 0 to 5 mM free Ca²⁺ in CB; and (g) a fluorescence spectrum and (h) a fluorescence titration curve for protonation depending on pH of FHEt-1_{lyso} under a condition of pH 4.3 to 11 in UB.

FIG. 7 is graphs showing two-photon (TP) fluorescence spectra of (a) BCa-1 in the presence of 0 to 39 μM free Ca²⁺; (b) BCa-2ₘᵢₜₒ in the presence of 0 to 2.5 mM free Ca²⁺; and (c) BCa−3°ₘₑₘ in the presence of 0 to 5 mM free Ca²⁺ in CB, (d) a TP fluorescence spectrum of FHEt-1_{lyso} under a condition of pH 4.3 to 11 in UB, (e) a TP fluorescence titration curve for complexation of Ca²⁺ and BCa-1 in the presence of 0 to 39 μM free Ca²⁺ in CB, and (0 a TP fluorescence titration curve for protonation of FHEt-1_{lyso} at pH 4.3 to 11 in UB.

FIG. 8 is graphs showing relative fluorescence intensities of (a) BCa-1, (b) BCa-2ₘᵢₜₒ, or (c) BCa−3°ₘₑₘ in the presence of 2 mM Mg²⁺ or 100 μM Zn²⁺, Mn²⁺, Fe²⁺, Cu²⁺, or Co²⁺ (black bars) and according to subsequent addition of 100 μM Ca²⁺ (red bars) in a MOPS buffer (30 mM 3[N-morpholino]propanesulfonic acid, 100 mM KCl, pH 7.2). At this time, excitation wavelengths are (a) 363 nm, (b) 355 nm, and (c) 350 nm, respectively.

FIG. 9 is graphs showing results of confirming effects of pH on one-photon fluorescence intensities of (a) BCa-1, (b) BCa-2ₘᵢₜₒ, or (c) BCa−3°ₘₑₘ in the presence of CaCl₂ (2.5 mM; red circles) and EDTA (2.5 mM; black squares) in an aqueous buffer (pH 4 to 10). At this time, excitation wavelengths are (a) 363 nm, (b) 355 nm, and (c) 350 nm, respectively.

FIG. 10 is a graph showing two-photon excitation spectra of BCa-1, BCa-2ₘᵢₜₒ, and BCa−3°ₘₑₘ in the presence or absence of excess Ca²⁺ in an aqueous buffer and a two-photon excitation spectrum of FHEt-1_{lyso} at pH 4.3 or 11.

FIG. 11 illustrates two-photon microscopy (TPM) images of HeLa cells labeled with (a) BCa-1-AM, (c) BCa-2ₘᵢₜₒ-AM, (e) BCa-3ₘₑₘ, or (g) FHEt-1_{lyso} (scale bar: 20 μm) and (b, d, f, and h) graphs showing relative TPEF intensities as a function of time for ROI′ to R013 of each image. At this time, the digitized fluorescence intensities were recorded at 2-second intervals for 1 hour in an xyt mode upon excitation at 750 nm.

FIG. 12 illustrates graphs showing results of measuring viability of HeLa cells after incubating for 2 hours or 24 hours according to a treatment concentration of each probe through CCK-8 analysis, with respect to HeLa cells treated with (a) BCa-1-AM, (b) BCa-2ₘᵢₜₒ-AM, (c) BCa-3ₘₑₘ, or (d) FHEt-1_{lyso}.

FIG. 13 illustrates graphs showing normalized TPEF spectra at an excitation wavelength of 750 nm with respect to HeLa cells labeled with (a) BCa-2ₘᵢₜₒ-AM and MitoTracker Red, (b) BLT-blue and FHEt-1_{lyso}, or (c) BCa-1-AM and FHEt-1_{lyso}.

FIG. 14 shows TPM images of Hela cells labeled with 0.5 μM BCa-3ₘₑₘ (top panel) or 0.5 μM BCa-3°ₘₑₘ (bottom panel) for 10 minutes at room temperature (scale bar: 20 μm). The images were captured at 10-minute intervals for 60 minutes upon excitation at 750 nm, and the brightness of the bottom image was increased by 100% compared to the top image.

FIG. 15 illustrates (a) a TPM image (scale bar: 10 μm) for Hela cells labeled with BCa-3ₘₑₘ and (b) a graph showing a change in relative TPEF intensity as a function of time for the ROI of the image. At this time, TPEF data were measured at 380 to 660 nm upon excitation at 750 nm.

FIG. 16 illustrates (a) a TPM image (scale bar: 20 μm) for Hela cells labeled with BCa-1-AM and (b) a graph showing a change in relative TPEF intensity as a function of time for the ROI of the image. At this time, TPEF data were measured at 380 to 660 nm upon excitation at 750 nm, and x and y axes of the graph mean 30 seconds and 10% of the change in fluorescence intensity.

FIG. 17 illustrates TPM images of Hela cells co-labeled with (a) BCa-2ₘᵢₜₒ-AM and (b) MitoTracker Red and (c) a TPM image obtained by merging these images; TPM images of Hela cells labeled with BCa-2ₘᵢₜₒ-AM at time points of (d) 0 second and (e) 1200 seconds; and (0 a graph showing a change in TPEF intensity of ROI 1 (mitochondria, black curve) and ROI 2 (cytoplasm, red curve) over time in HeLa cells labeled with BCa-2ₘᵢₜₒ-AM. At this time, each TPM image was captured at 380 to 540 nm (a, d, e: BCa-2ₘᵢₜₒ-AM) and 600 to 680 nm (b: MitoTracker Red) upon excitation at 750 nm, and the scale bars were 10 μm (a to c) and 20 μm (d, e).

FIG. 18 shows TPM images of Hela cells labeled with (a) BLT-blue and (b) FHEt-1_{lyso}, and (c) a TPM image obtained by merging these images. At this time, each TPM image was captured at 380 to 480 nm (a: BLT-blue) and 550 to 660 nm (b: FHEt-1 lyso) upon excitation at 750 nm, A is Pearson's colocalization coefficient, and the scale bar is 20 μm.

FIG. 19 illustrates TPM images of HeLa cells co-labeled with BCa-1 and FHEt-1_{lyso} at time points of (a, b) 0 second and (c, d) 700 seconds; (e) a TPM image obtained by merging the images of (a) and (b); and (f, g) graphs showing changes in TPEF intensity as a function of time for the ROI of the image of (e) above. At this time, the cells were added with monensin at 100 seconds, and TPEF was measured at 380 to 480 nm (a, c: BCa-1) and 550 to 660 nm (b, d: FHEt-1_{lyso}) upon excitation at 750 nm, and the scale bar is 10 μm.

FIG. 20 illustrates (a) a dual-color cross-sectional TPM image at depths of 90 to 140 μm (10× magnification) of rat hippocampal slices co-labeled with BCa-1-AM and FHEt-1_{lyso}; (b) a dual-color cross-sectional TPM image at a depth of 100 μm of the panel (a) above; images photographed at (c) 380 to 480 nm (cytoplasm) and (d) 550 to 660 nm (lysosome) by magnifying a white rectangular area of the panel (b) above; (e) an image obtained by merging the images of (c) and (d) above; TPM images captured at (f) 100× magnification or (g) 100× magnification at 4x zoom at 100 μm depth for rat hippocampal slices labeled with BCa-2 onto-AM; and TPM images captured at (h) 100× magnification or (i) 100× magnification at 4x zoom at 100 μm depth for rat hippocampal slices labeled with BCa-3ₘₑₘ. At this time, each image was photographed upon excitation at 750 nm, and the scale bars are (b) 100 μm, (c to e, g, i) 5 μm, and (f, h) 20 μm.

FIG. 21 illustrates cross-sectional TPM images at depths of 90 to 140 μm (10× magnification) of rat hippocampal slices labeled with (a) BCa-2ₘᵢₜₒ-AM or (b) BCa-3ₘₑₘ. At this time, the images were shown by measuring emission at 380 to 660 nm upon excitation at 750 nm.

FIG. 22 illustrates a 1H NMR spectrum (500 MHz, DMSO-d₆) and a ¹³C NMR spectrum (151 MHz, DMSO-d₆) of BCa-1.

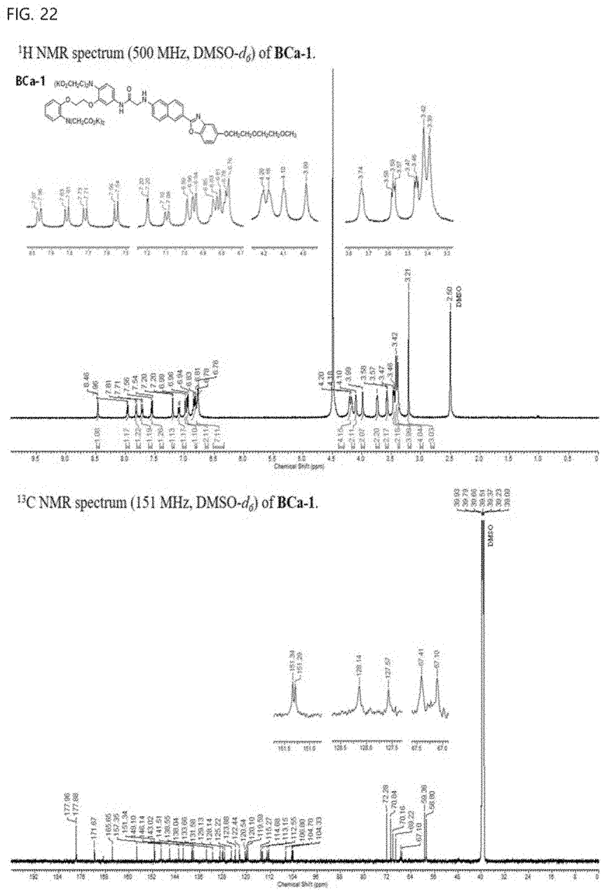

FIG. 23 illustrates a 1H NMR spectrum (500 MHz, CDCl₃) and a ¹³C NMR spectrum (151 MHz, CDCl₃) of BCa-1-AM.

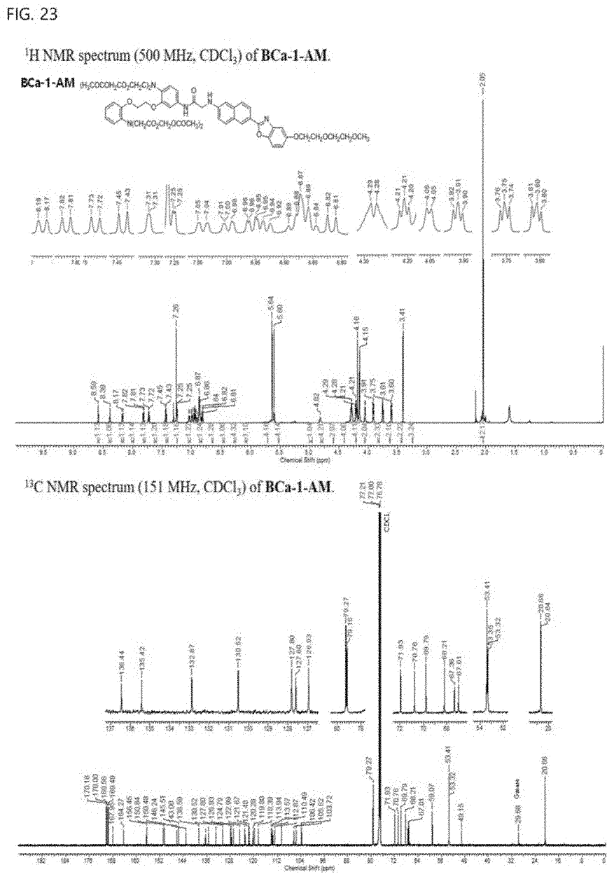

FIG. 24 illustrates a 1H NMR spectrum (500 MHz, CD₃CN) and a ¹³C NMR spectrum (75 MHz, CDCl₃) of BCa-2ₘᵢₜₒ-AM.

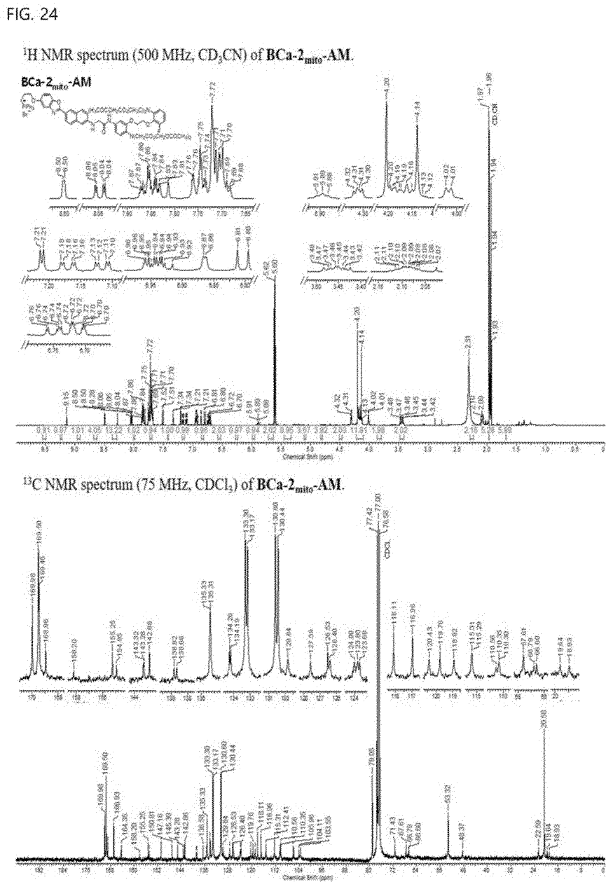

FIG. 25 illustrates a 1H NMR spectrum (500 MHz, DMSO-d₆/CD₃CN at 9: 1) and a ¹³C NMR spectrum (75 MHz, DMSO-d₆) of BCa-3ₘₑₘ.

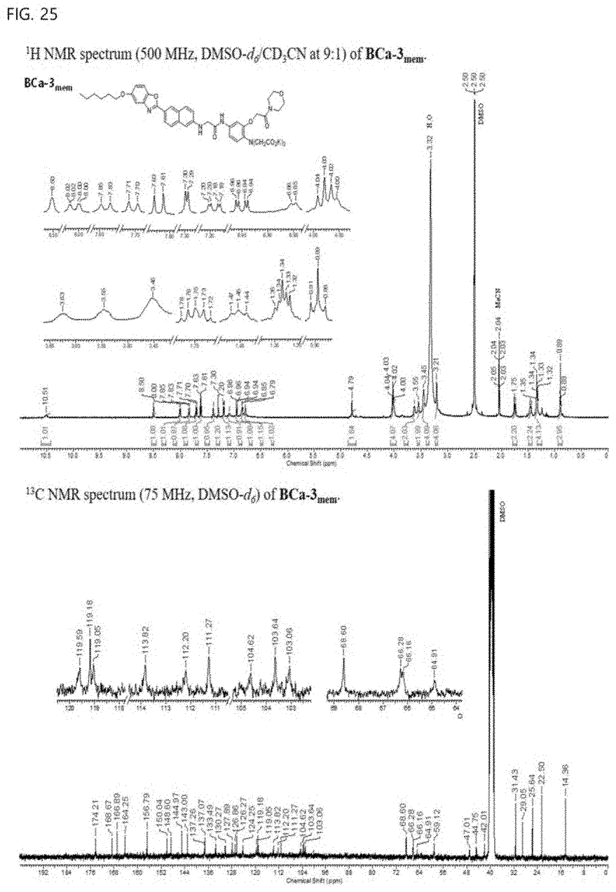

FIG. 26 illustrates a 1H NMR spectrum (500 MHz, DMSO-d₆) and a ¹³C NMR spectrum (151 MHz, DMSO-d₆) of BCa-3°ₘₑₘ.

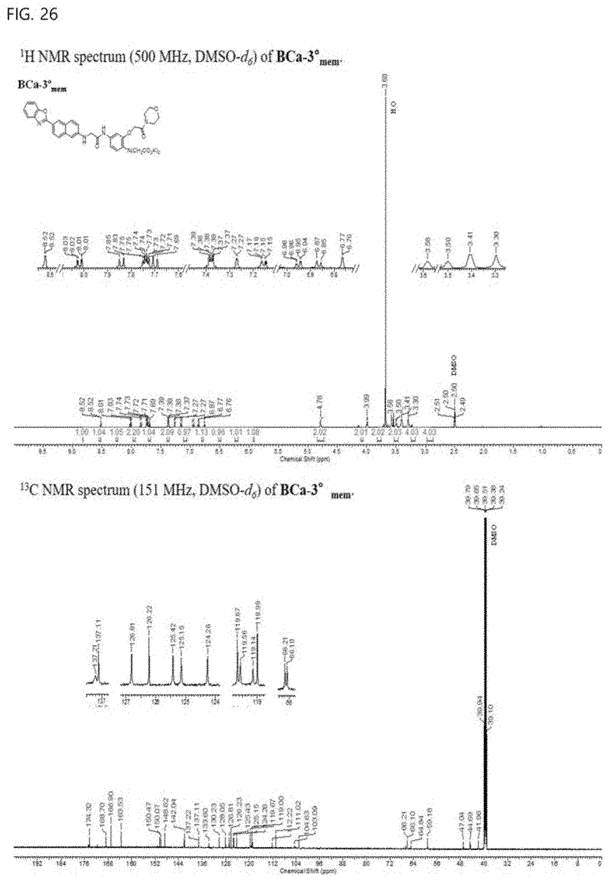

FIG. 27 illustrates a 1H NMR spectrum (600 MHz, CDCl₃) and a ¹³C NMR spectrum (75 MHz, CDCl₃) of FHEt-1_{lyso}.

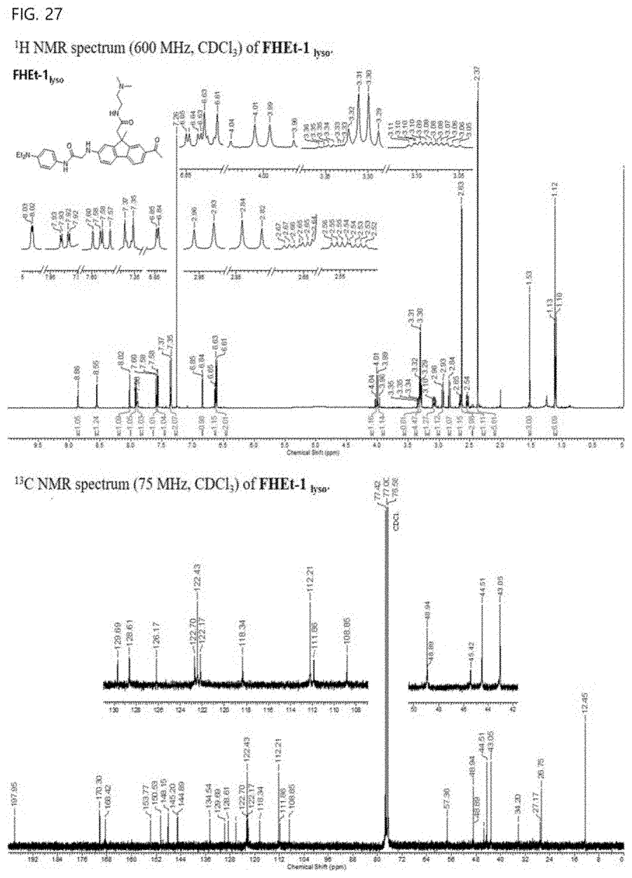

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in detail.

In order to design a two-photon (TP) probe (marker) for detecting Ca²⁺ for multicolor imaging, the present inventors developed organelles-specific blue emission TP probes BCa-1-AM, BCa-2ₘᵢₜₒ-AM, and BCa-3ₘₑₘ for Ca²⁺ by using 6-(benzo[d]oxazol-2-yl)-2-naphthalylamine (compound B) as a fluorophore and 0,0′-bis(2-aminophenyl)ethyleneglycol-N,N,N′,N′-tetraacetic acid (BAPTA) and 2-(2′-morpholino-2′-oxoethoxy)-N,N-bis(hydroxycarbonylmethyl)aniline (MOBHA) derivatives as Ca²⁺ receptors. Meanwhile, since BCa-3ₘₑₘ has too low aqueous solubility and its photophysical properties cannot be accurately measured in an aqueous buffer, the present inventors further designed BCa-3°ₘₑₘ to mimic BCa-3ₘₑₘ. In addition, the present inventors developed a TP probe FHEt-1_{lyso} for detecting lysosomal H⁺ by using 2-acetyl-7-aminofluorene (Compound F) as a green emission TP fluorophore and 4-diethylaminophenyl group as an H⁺ receptor. The present inventors confirmed that the prepared TP probes for detecting Ca²⁺ had high selectivity for specific organelles (cytoplasm, mitochondria, plasma membrane, respectively) and calcium ions, confirmed that the TP probe for detecting Ca²⁺ and the TP probe for detecting lysosomal H⁺ could be used together to simultaneously monitor changes in cytoplasmic Ca²⁺ and lysosomal 1-1+ concentrations in live cells and tissues in real time by dual-color TP microscopy, and then completed the present disclosure.

At this time, the compounds B and F were prepared by deriving from BLT-blue targeting the lysosome and FMT-green targeting mitochondria, respectively. The BLT-blue and FMT-green trackers have maximum TP excited fluorescence (TPEF) at 451 and 523 nm, respectively, and have high TP action cross-section values (Φδ_{eff}) of 2120×and 1480×10⁻⁵⁰ cm⁴s/photon (GM). In previous studies, it is confirmed that these trackers enable dual-color imaging of two organelles in probe-labeled cells.

In one aspect, the present disclosure provides a two-photon fluorescent probe compound represented by any one of Chemical Formulas 1 to 3 below:

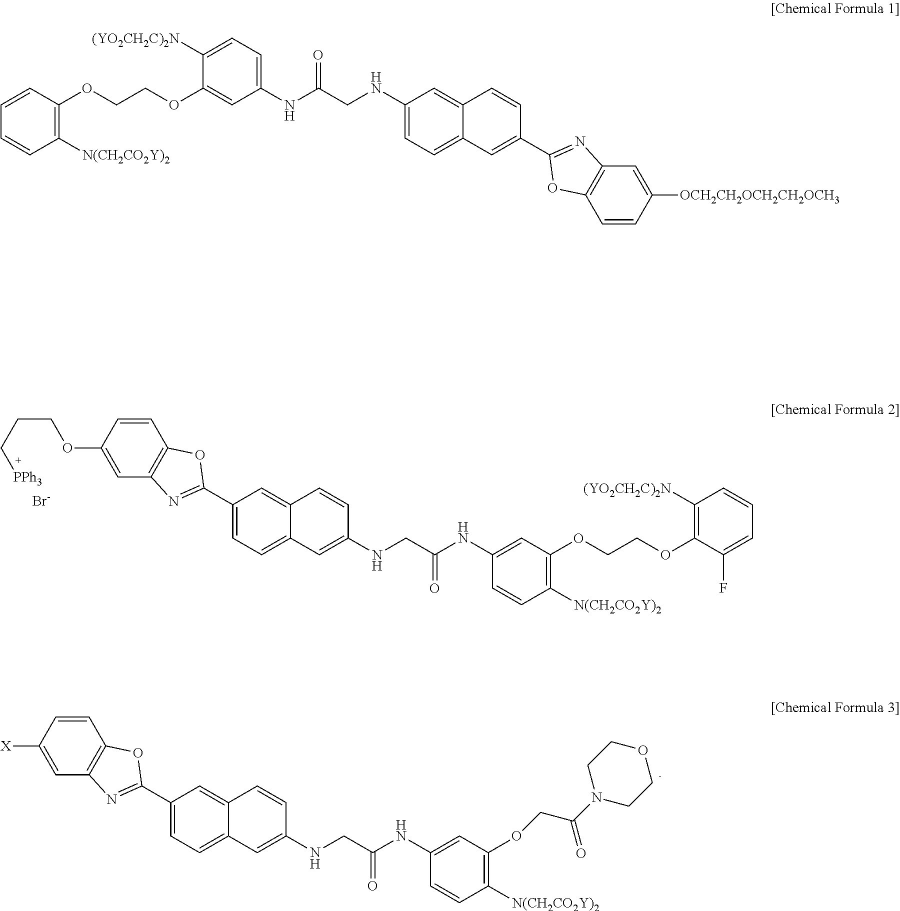

In Chemical Formulas 1 to 3, Y is each independently K, Na, CH₃, or CH₂OC(O)CH₃, and in Chemical Formula 3, X is H, OH, or O(CH₂)₅CH₃.

Preferably, in Chemical Formula 1, Y may be K or CH₂OC(O)CH₃.

Preferably, in Chemical Formula 2, the Y may be K or CH₂OC(O)CH₃.

Preferably, in Chemical Formula 3, Y may be K or CH₃, most preferably K, and X may be H or O(CH₂)₅CH₃.

According to one embodiment of the present disclosure, the compound represented by Chemical Formula 1 may be a compound represented by [BCa-1] or [BCa-1-AM] below:

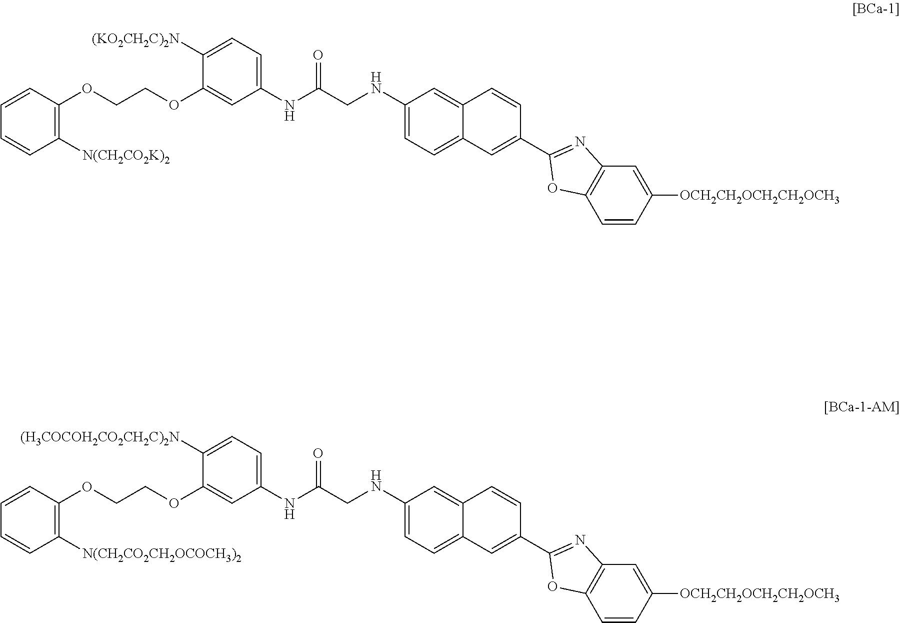

According to one embodiment of the present disclosure, the compound represented by Chemical Formula 2 may be a compound represented by [BCa-2-ₘᵢₜₒ] or [BCa-2-mito-AM] below:

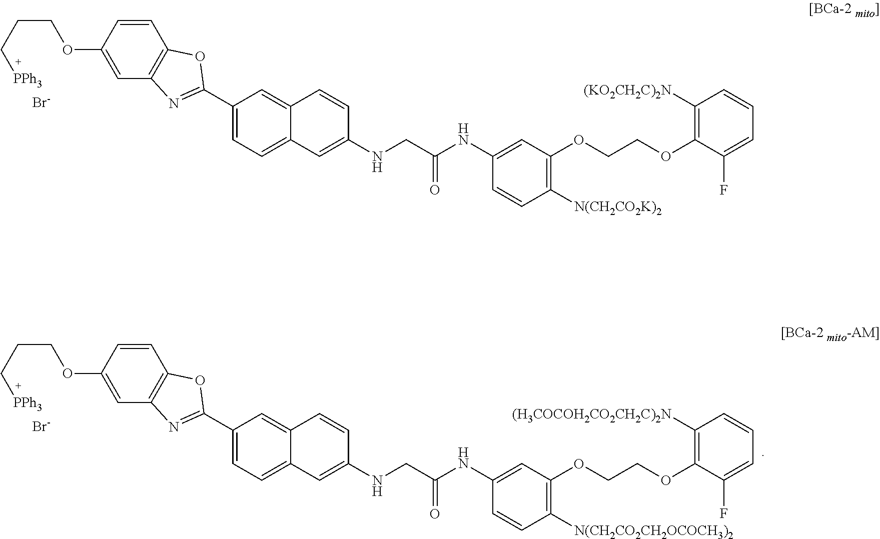

According to one embodiment of the present disclosure, the compound represented by Chemical Formula 3 may be a compound represented by [BCa-3ₘₑₘ] or [BCa-3°ₘₑₘ] below:

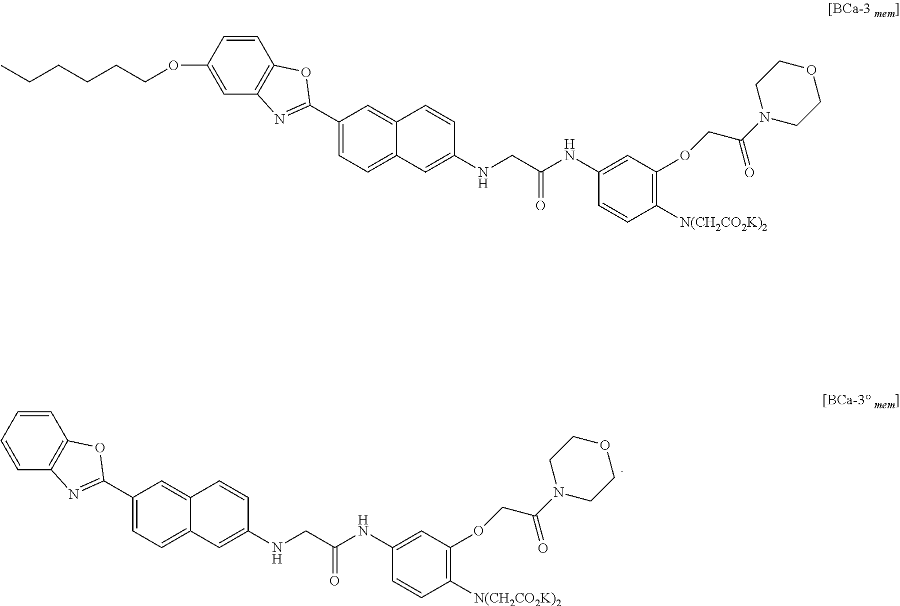

The two-photon fluorescent probe compound represented by any one of Chemical Formulas 1 to 3 according to the present disclosure may specifically bind to calcium ions (Ca²⁺), may react with calcium ions to form a complex, and may emit blue fluorescence with a fluorescence reaction to calcium ions.

The two-photon fluorescent probe compound represented by any one of Chemical Formulas 1 to 3 according to the present disclosure may be for selectively imaging calcium ions in the cytoplasm, mitochondria, or plasma membrane.

According to one embodiment of the present disclosure, the two-photon fluorescent probe compound represented by Chemical Formula 1, the two-photon fluorescent probe compound represented by Chemical Formula 2, and the two-photon fluorescent probe compound represented by Chemical Formula 3 may be for selectively imaging calcium ions in the cytoplasm, calcium ions in the mitochondria, and calcium ions in the plasma membrane, respectively.

In addition, the present disclosure provides a two-photon fluorescent probe composition for detecting calcium ions in live cells or tissues including at least one compound selected from the two-photon fluorescent probe compounds represented by any one of Chemical Formulas 1 to 3.

The two-photon fluorescent probe composition for detecting calcium ions according to the present disclosure may be selectively imaging calcium ions in the cytoplasm, mitochondria, and/or plasma membrane. The depth of the imaging may be 90 to 140 μm.

The two-photon fluorescent probe composition for detecting calcium ions according to the present disclosure may also include only one compound among the compounds represented by Chemical Formula 1, 2, or 3, or a mixture of two compounds, and may also include all three compounds represented by Chemical Formulas 1, 2, and 3, respectively.

In one embodiment, the composition may be a two-photon fluorescent probe composition for detecting calcium ions in live cells or tissues including a compound represented by Chemical Formula 1 (preferably, [BCa-1] and/or [BCa-1-AM]), a compound represented by Chemical Formula 2 (preferably [BCa-2ₘᵢₜₒ] and/or [BCa-2ₘᵢₜₒ-AM]), and a compound represented by Chemical Formula 3 (preferably [BCa-3ₘₑₘ] and/or [BCa−3°ₘₑₘ]). The composition may be for simultaneously imaging calcium ions located in organelles, preferably the cytoplasm, mitochondria, and plasma membrane, respectively, and for simultaneously monitoring changes in distribution or concentration of calcium ions in the cytoplasm, mitochondria, and plasma membrane, respectively.

The two-photon fluorescent probe composition for detecting calcium ions according to the present disclosure may further include at least one fluorescent probe compound selected from the group consisting of MitoTracker Red, BLT-blue, and FMT-green.

Further, the present disclosure provides a method for imaging calcium ions including injecting at least one compound selected from the two-photon fluorescent probe compounds represented by any one of Chemical Formulas 1 to 3 into cells or tissues isolated from a living body; irradiating an excitation source to the cells or tissues isolated from the living body; and observing fluorescence generated from the two-photon fluorescent probe compound by two-photon microscopy.

The method for imaging calcium ions according to the present disclosure may target the cytoplasm, mitochondria, and/or plasma membrane to selectively image calcium ions in the cytoplasm, mitochondria, and/or plasma membrane. The depth of the imaging may be 90 to 140 μm.

In the method for imaging calcium ions according to the present disclosure, the injecting step may be injecting at least one compound itself selected from the two-photon fluorescent probe compounds represented by any one of Chemical Formulas 1 to 3, and may be injecting the composition containing at least one compound selected from the two-photon fluorescent probe compounds represented by any one of Chemical Formulas 1 to 3, for example, the two-photon fluorescent probe composition for detecting calcium ions.

In addition, it is possible to monitor changes in calcium distribution or concentration in live cells or tissues using the method for imaging calcium ions according to the present disclosure, and it is also possible to quantitatively measure the changes as well as qualitative analysis.

In addition, in the method for imaging calcium ions according to the present disclosure, the injecting step may be further injecting at least one fluorescent probe compound selected from the group consisting of MitoTracker Red, BLT-blue, and FMT-green, and it is possible to simultaneously monitor changes in calcium distribution or concentration in each organelle using the same.

In addition, the present disclosure provides a method for preparing a two-photon fluorescent probe compound represented by any one of Chemical Formulas 1 to 3 below, including reacting a compound represented by Chemical Formula 5 below with a compound represented by Chemical Formula 6 or 7 below:

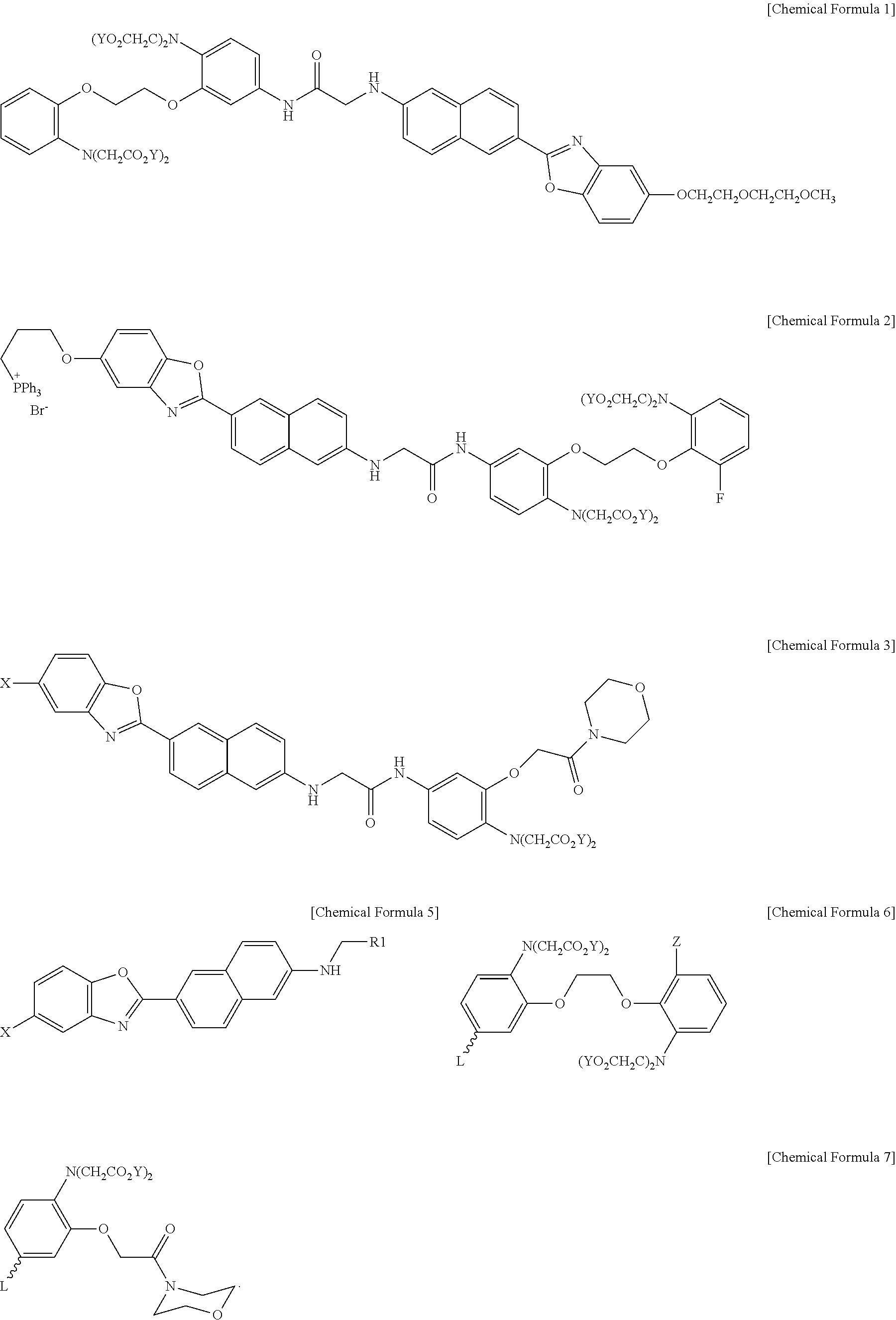

In Chemical Formula 1, 2, 3, 5, 6, or 7, independently of each other, Y is Na, K, Na, CH₃, or CH₂OC(O)CH₃, X is H, OH, O(CH₂CH₂O)₂CH₃, O(CH₂)₃Br, O(CH₂)₃PPh₃⁺Br⁻, or O(CH₂)₅CH₃, R1 is CO₂C(CH₃)₃ or CO₂H, Z is H or F, and L is H or NH₂.

Preferably, the method for preparing the compounds represented by Chemical Formulas 1 and 2 may include reacting the compound represented by Chemical Formula 5 with the compound represented by Chemical Formula 6, and the method for preparing the compound represented by Chemical Formula 3 may include reacting the compound represented by Chemical Formula 5 with the compound represented by Chemical Formula 7.

In one embodiment, in Chemical Formula 1, Y is K or CH₂OC(O)CH₃, in Chemical Formula 5, X is OH, R1 is CO₂H, and in Chemical Formula 6, Y is CH₃, Z is H, and L may be NH₂. At this time, the method for preparing the two-photon fluorescent probe compound represented by Chemical Formula 1 may further include substituting X in Chemical Formula 5 with O(CH₂CH₂O)₂CH₃ and substituting CH₃ corresponding to Y in Chemical Formula 6 with K or CH₂OC(O)CH₃ corresponding to Y in Chemical Formula 1.

In one embodiment, in Chemical Formula 2, Y is K or CH₂OC(O)CH₃, in Chemical Formula 5, X is OH and R1 is CO₂H, and in Chemical Formula 6, Y is CH₃, Z is F, and L may be NH₂. At this time, the method for preparing the two-photon fluorescent probe compound represented by Chemical Formula 2 may further include substituting the OH with O(CH₂)₃Br or O(CH₂)₃PPh₃⁺Br⁻ when X in Chemical Formula 5 is OH, and further include substituting the CH₃ with K or CH₂OC(O)CH₃ when Y in Chemical Formula 6 is CH₃.

In one embodiment, in Chemical Formula 3, Y is K or CH₃ and X is O(CH₂)₅CH₃ or H, in Chemical Formula 5, X is H or OH and R1 is CO₂H, and in Chemical Formula 7, Y is CH₃ and L may be NH₂. At this time, the method for preparing the two-photon fluorescent probe compound represented by Chemical Formula 3 may further include substituting the OH with O(CH₂)₅CH₃ when X in Chemical Formula 5 is OH, and further include substituting the CH₃ with K or CH₂OC(O)CH₃ when Y in Chemical Formula 7 is CH₃.

In one embodiment, the two-photon fluorescent probe compound represented by any one of Chemical Formulas 1 to 3 may be prepared by a method including Reaction Formula 1 below, but is not limited thereto.

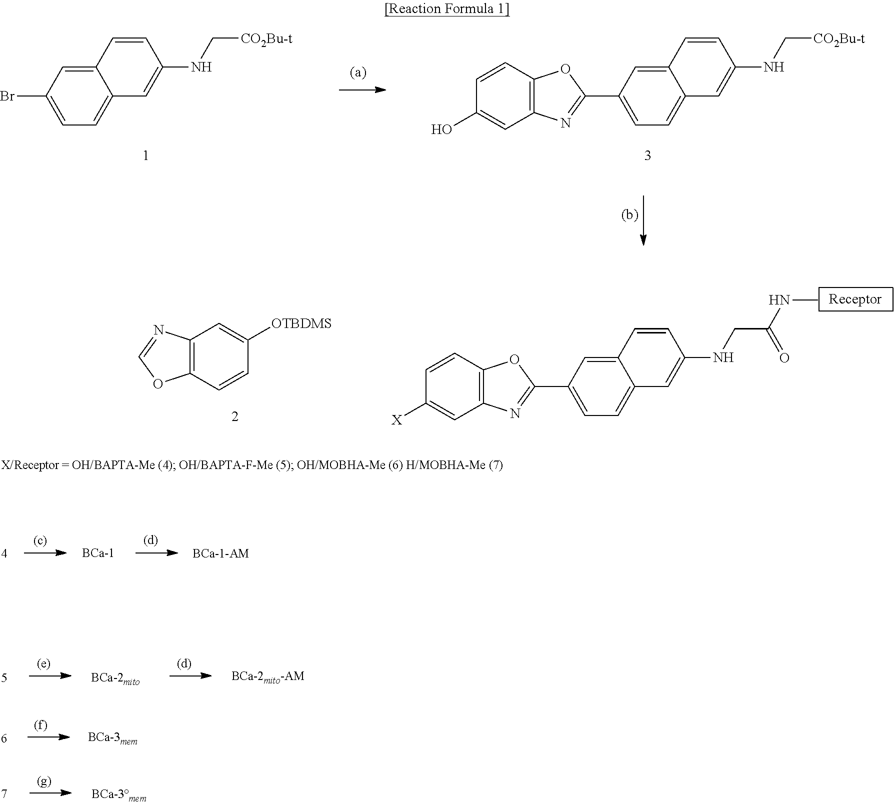

In Reaction Formula 1, reaction conditions may be (a) Compound 2, Pd(OAc)₂, CuI, Cs₂CO₃, Xphos, dimethylformamide (DMF), 120° C.; (b) (i) trifluoroacetic acid, CH₂Cl₂, RT; (ii) HATU, (i-Pr)₂NEt, H₂N-BAPTA-Me, DMF, RT; (c) (i) Me(OCH₂CH₂)₂OTs, K₂CO₃, DMF, 120° C.; (ii) KOH, EtOH/dioxane, 0° C.; (d) BrCH₂OC(0)Me, (i-Pr)₂NEt, MeCN/DMF, RT; (e) (i) BrCH₂CH₂CH₂Br, K₂CO₃, DMF, 75° C.; (ii) PPh₃, MeCN, 90° C.; (iii) KOH, EtOH/dioxane, RT; (f) n-C₆H₁₃Br, K₂CO₃, DMF, 80° C.; (ii) KOH, EtOH/dioxane, 0° C.; and (g) KOH, EtOH/dioxane, 0° C. The two-photon fluorescent probe compound represented by Chemical Formula 1 prepared by the method including Reaction Formula 1 may be BCa-1 or BCa-1-AM, the two-photon fluorescent probe compound represented by Chemical Formula 2 prepared by the method including Reaction Formula 1 may be the BCa-2ₘᵢₜₒ or BCa-2ₘᵢₜₒ-AM, and the two-photon fluorescent probe compound represented by Chemical Formula 3 prepared by the method including Reaction Formula 1 may be the BCa-3ₘₑₘ or BCa-3°ₘₑₘ.

In one aspect, the present disclosure provides a two-photon fluorescent probe compound represented by Chemical Formula 4 below:

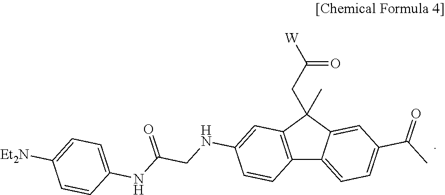

In Chemical Formula 4, W is NH(CH₂)₂N(CH₃)₂ or OCH₃. Preferably, W may be NH(CH₂)₂N(CH₃)₂.

According to one embodiment of the present disclosure, the compound represented by Chemical Formula 4 may be a compound represented by [FHEt-1_{lyso}]:

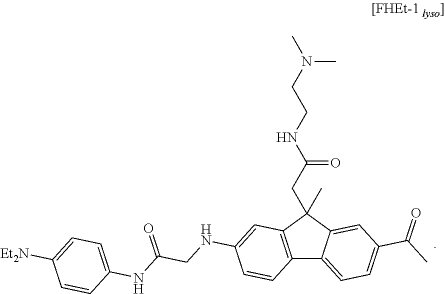

The two-photon fluorescent probe compound represented by Chemical Formula 4 according to the present disclosure may specifically bind to hydrogen ions (H⁺) and emit green fluorescence by a fluorescence reaction with hydrogen ions.

The two-photon fluorescent probe compound represented by Chemical Formula 4 according to the present disclosure may be for selectively imaging hydrogen ions in the lysosome or for imaging changes in acidity (pH) in the lysosome.

In addition, the present disclosure provides a two-photon fluorescent probe composition for detecting hydrogen ions in live cells or tissues, including the two-photon fluorescent probe compound represented by Chemical Formula 4.

The two-photon fluorescent probe composition for detecting hydrogen ions according to the present disclosure may selectively image hydrogen ions in the lysosome, and the depth of the imaging may be 90 to 140 μm.

Further, the present disclosure provides a method for imaging hydrogen ions including injecting the two-photon fluorescent probe compound represented by Chemical Formula 4 into cells or tissues isolated from a living body; irradiating an excitation source to the cells or tissues isolated from the living body; and observing fluorescence generated from the two-photon fluorescent probe compound by two-photon microscopy.

The method of imaging hydrogen ions according to the present disclosure may be selectively imaging hydrogen ions in the lysosome by targeting the lysosome.

In addition, it is possible to monitor changes in distribution or concentration of hydrogen ions in live cells or tissues using the method for imaging the hydrogen ions according to the present disclosure, and it is also possible to quantitatively measure the changes as well as qualitative analysis and to monitor a change in pH.

In addition, the present disclosure provides a method for preparing a two-photon fluorescent probe compound represented by Chemical Formula 4 below, including reacting a compound represented by Chemical Formula 8 below with a compound represented by Chemical Formula 9 below:

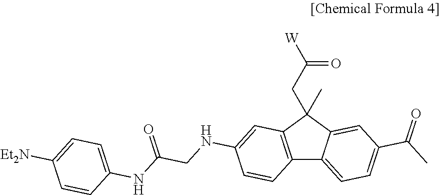

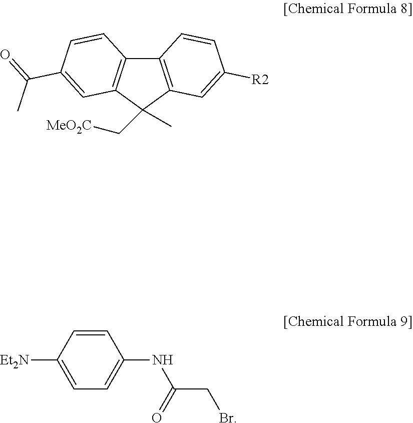

In Chemical Formula 4, W is NH(CH₂)₂N(CH₃)₂ or OCH₃, and in Chemical Formula 8, R2 is NO₂ or NH₂.

Preferably, in Chemical Formula 4, W may be NH(CH₂)₂N(CH₃)₂, and in Chemical Formula 8, R2 may be NH₂. At this time, the method for preparing the two-photon fluorescent probe compound represented by Chemical Formula 4 according to the present disclosure may further include substituting CO₂Me in Chemical Formula 8 with CO₂NH(CH₂)₂N(CH₃)₂.

In one embodiment, the two-photon fluorescent probe compound represented by Chemical Formula 4 may be prepared by a method including Reaction Formula 2 below, but is not limited thereto.

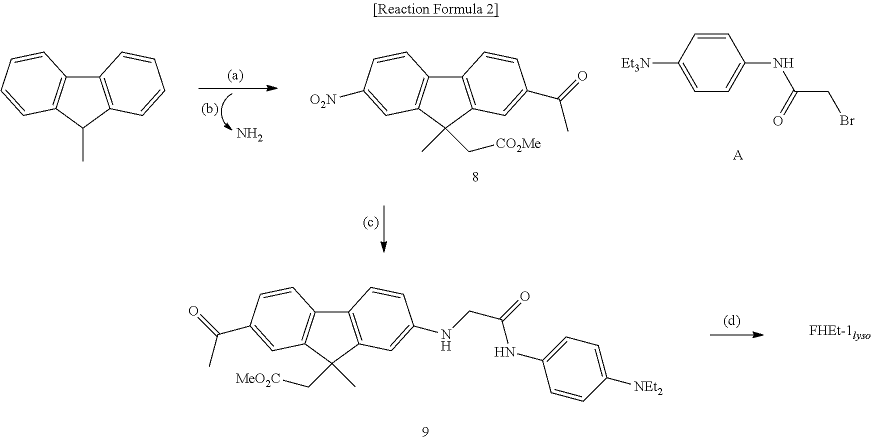

In Reaction Formula 2, the reaction conditions may be (a) (i) BrCH₂CO₂Me, NaOtBu, dimethyl sulfoxide; (ii) HNO₃, CH₂Cl₂, 0° C.; (iii) MeC(O)Cl, AlCl₃, nitrobenzene, 60° C.; (b) Fe, NH₄Cl(aq), EtOH/dioxane, reflux; (c) Compound A (compound represented by Chemical Formula 9), (i-Pr)₂NEt, DMF; and (d) (i) KOH, EtOH, RT; (ii) N,N-dimethylethylenediamine, HATU, (i-Pr)₂NEt, DMF, RT.

In one aspect, the present disclosure provides a two-photon fluorescent probe composition for simultaneously detecting calcium ions and hydrogen ions including at least one compound selected from two-photon fluorescent probe compounds represented by any one of Chemical Formulas 1 to 3 below and a compound represented by Chemical Formula 4 below:

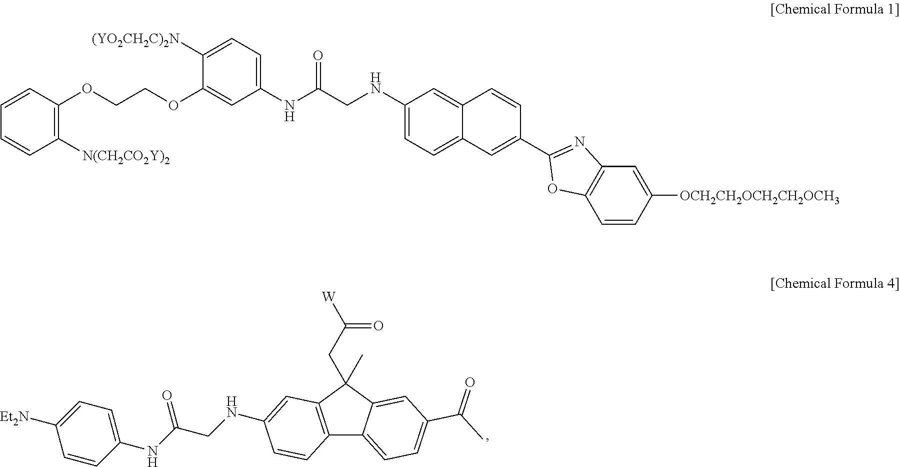

In Chemical Formulas 1 to 3, Y is each independently K, Na, CH₃, or CH₂OC(O)CH₃, in Chemical Formula 3, X is H, OH or O(CH₂)₅CH₃, and in Chemical Formula 4, W is NH(CH₂)₂N(CH₃)₂ or OCH₃.

Preferably, in Chemical Formula 1, Y is K or CH₂OC(O)CH₃, in Chemical Formula 2, Y is K or CH₂OC(O)CH₃, in Chemical Formula 3, Y is K, and X is H or O(CH₂)₅CH₃, and in Chemical Formula 4, W may be NH(CH₂)₂N(CH₃)₂.

According to one embodiment of the present disclosure, the compound represented by Chemical Formula 1 may be the compound represented by [BCa-1] or [BCa-1-AM], the compound represented by Chemical Formula 2 may be the compound represented by [BCa-2-ₘᵢₜₒ] or [BCa-2-ₘᵢₜₒ-AM], the compound represented by Chemical Formula 3 may be the compound represented by [BCa-3ₘₑₘ] or [BCa-3°ₘₑₘ], and the compound represented by Chemical Formula 4 may be the compound represented by [FHEt-1_{lyso}].

In the two-photon fluorescent probe composition for simultaneously detecting calcium ions and hydrogen ions according to the present disclosure, the two-photon fluorescent probe compound represented by any one of Chemical Formulas 1 to 3 may target the cytoplasm, mitochondria, or plasma membrane to selectively image calcium ions in the cytoplasm, mitochondria, or plasma membrane, and the compound represented by Chemical Formula 4 may target the lysosome to selectively image hydrogen ions in the lysosome.

Preferably, the compound of Chemical Formula 1 may target the cytoplasm to selectively image calcium ions in the cytoplasm, the compound of Chemical Formula 2 may target the mitochondria to selectively image calcium ions in the mitochondria, the compound of Chemical Formula 3 may target the plasma membrane to selectively image calcium ions in the plasma membrane, and the compound of Chemical Formula 4 may target the lysosome to selectively image hydrogen ions in the lysosome.

Further, the present disclosure provides a method for simultaneously imaging calcium ions and hydrogen ions including injecting at least one compound selected from the two-photon fluorescent probe compounds represented by any one of Chemical Formulas 1 to 3 and the two-photon fluorescent probe compound represented by Chemical Formula 4 into cells or tissues isolated from a living body; irradiating an excitation source to the cells or tissues isolated from the living body; and observing fluorescence generated from the two-photon fluorescent probe compounds by two-photon microscopy.

In addition, by using the method of simultaneously imaging calcium ions and hydrogen ions according to the present disclosure, it is possible to monitor changes in distribution or concentration of calcium ions in the cytoplasm, mitochondria, and/or plasma membrane and hydrogen ions in the lysosome in live cells or tissues and also to measure quantitatively the changes as well as qualitative analysis.

In the method of simultaneously imaging calcium ions and hydrogen ions according to the present disclosure, the imaging may be simultaneously imaging changes in distribution of calcium ions in the cytoplasm, mitochondria, and/or plasma membrane and pH of the lysosome.

In one embodiment of the present disclosure, it was confirmed that organelles-specific blue emission TP probes BCa-1-AM, BCa-2ₘᵢₜₒ-AM, and BCa-3ₘₑₘ for Ca²⁺ prepared by the method according to the present disclosure had a maximum absorption (λₘₐₓ) of 350 to 358 nm, a maximum emission (λ_{fl}) of 464 to 466 nm, and a TP action cross-section (Φδₘₐₓ) of 55 to 70×10⁻⁵⁰ cm⁴s/photon at 750 nm in the presence of excess Ca²⁺. It was confirmed that the TP probes had dissociation constants of 0.18, 2.7, and 100 μM, which were appropriate values for detecting Ca²⁺ in the cytoplasm, mitochondria, and plasma membrane (cell membrane), respectively, and it was confirmed through TP microscopic analysis that the TP probes facilitated real-time detection of Ca²⁺ in the cytoplasm, mitochondria, and plasma membrane of live cells and tissues. In addition, it was confirmed that the green emission TP probe FHEt-1_{lyso} for lysosomal H⁺ prepared by the method according to the present disclosure had λₘₐₓ of 359 nm, λ_{fl} of 571 nm, and Φδₘₐₓ of 70×10⁻⁵⁰ cm⁴s/photon in a universal buffer at pH 4.3, and it was confirmed through TP microscopy analysis that the TP probe facilitated the detection of H⁺ in the lysosome. Specifically, it was confirmed that by using BCa-1-AM (or BCa-1) and FHEt-1_{lyso}, it is possible to simultaneously monitor changes in cytoplasmic Ca²⁺ and lysosomal H⁺ concentrations in live cells and tissues (or Hela cells) in real time by dual-color TP microscopy.

Hereinafter, preferred Examples will be proposed in order to help in understanding of the present disclosure. However, the following Examples are just provided to more easily understand the present disclosure and the contents of the present disclosure are not limited by Examples.

Preparation Example 1. Devices and Materials

¹H NMR and ¹³C NMR spectra were measured by using CDCl₃, (CD₃)₂₅₀, CD₃OD, or CD₃CN as a solvent and internal reference [CDCl₃: δ H 7.26, δ C 77.0; (CD₃)₂SO: δ H 2.50, δ C 39.51; CD₃CN: δ H 1.94, δ C 1.39] in Varian 300 (300 MHz for ¹H NMR and 75 MHz for ¹³C NMR) and Bruker Avance III 500 and 600 (500 and 600 MHz for ¹H NMR and 151 MHz for ¹³C NMR) spectrometers.

High-resolution mass spectra (HRMS) were analyzed using AB SCIEX Triple TOF® 5600 plus (for BCa-1, BCa-2 onto-AM, BCa-3ₘₑₘ, BCa−3°ₘₑₘ and FHEt-1_{lyso}) and Thermo Scientific Q Exactive (for BCa-1-AM and BCa-2 onto) mass spectrometry.

All chemical reagents were purchased from commercial suppliers and used without further purification unless otherwise specified.

### EXAMPLE 1. Preparation of Two-Photon Probe

Compound 3°, Compound C2, BAPTA-Me, BAPTA-F-Me, and MOBHA-Me were synthesized according to the related art [Analytical Chemistry (2012) 84:8110-8113, Angewandte Chemie International Edition (2013) 52:3874-3877, Nature Chemical Biology (2007) 3:423-431]. Synthesis of other compounds was shown in Reaction Formulas 1-1 and 1-2 below.

First, BCa-1, BCa-1-AM, BCa-2ₘᵢₜₒ, BCa-2 onto-AM, BCa-3ₘₑₘ, and BCa-3°ₘₑₘ were synthesized by a method of Reaction Formula 1-1 below.

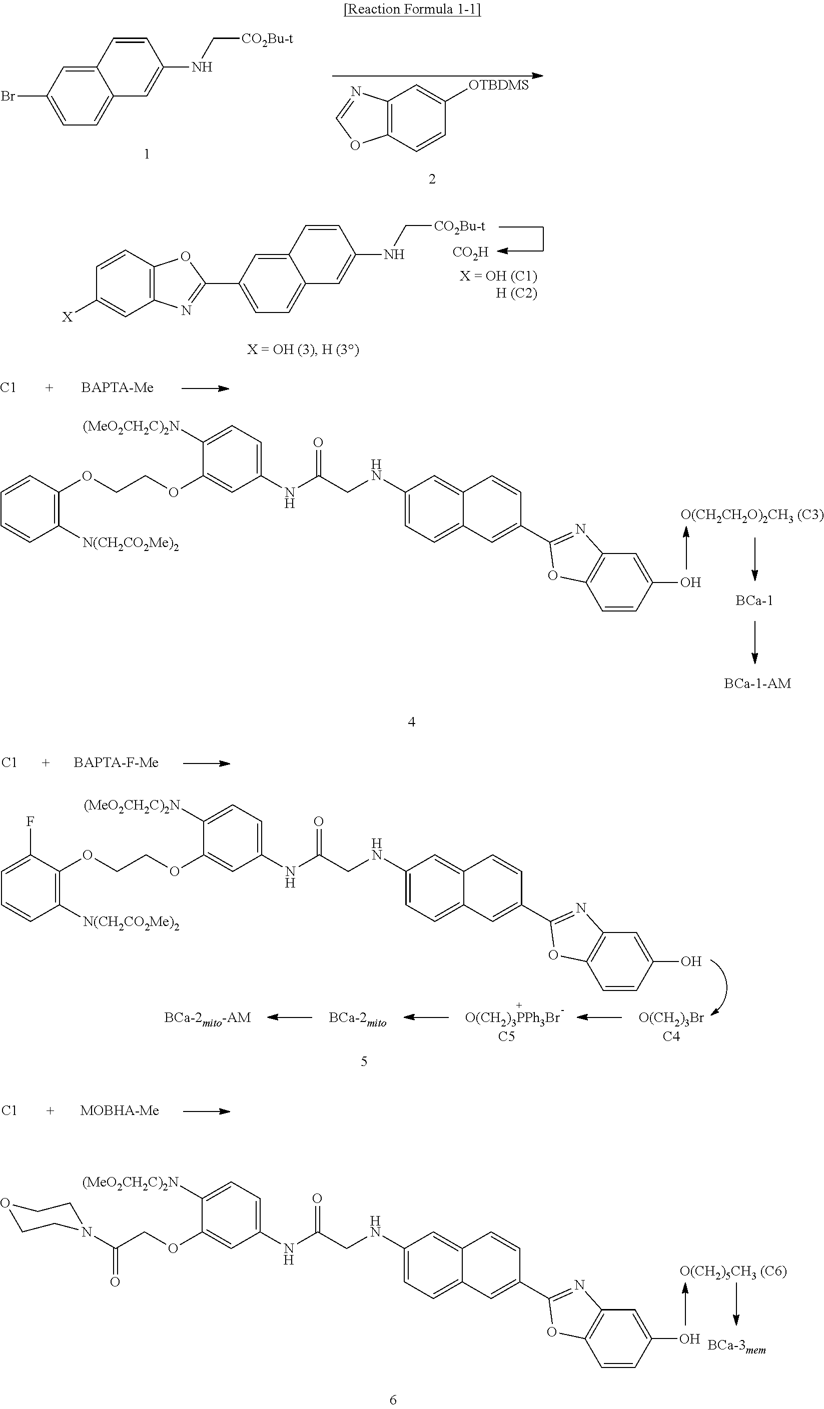

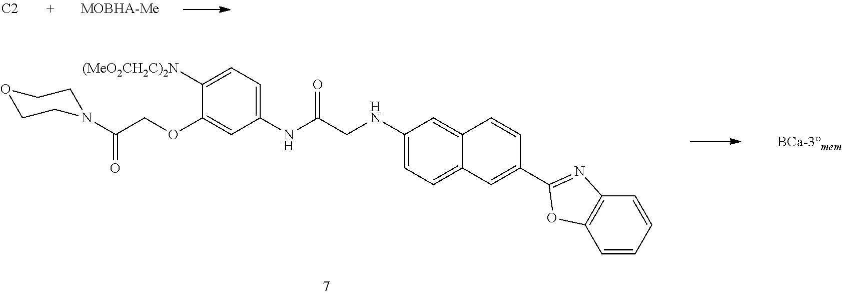

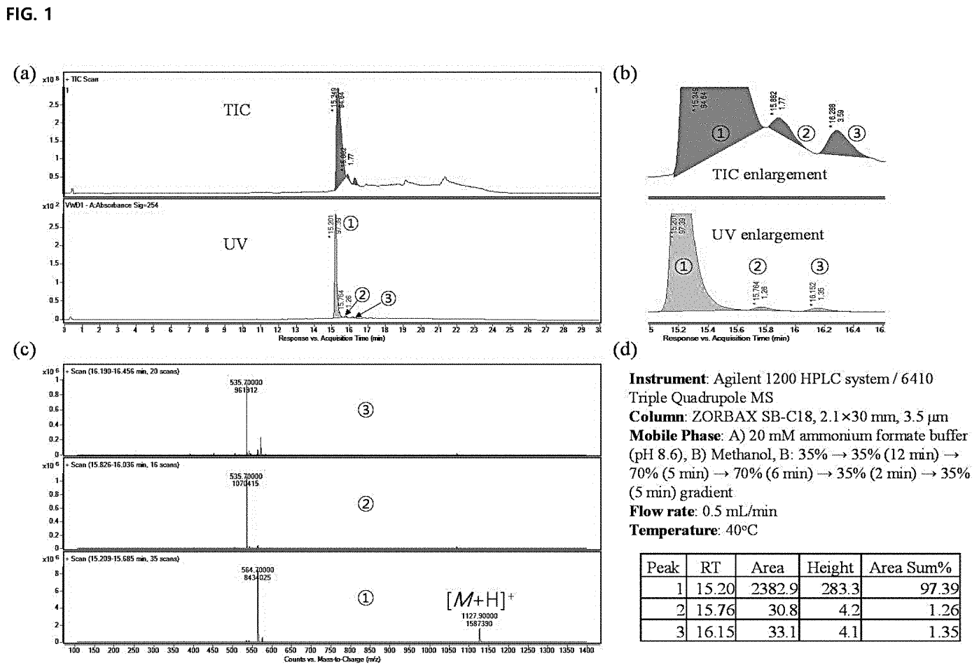

Briefly, Compound 3 was prepared by combining Compound 1 and Compound 2 in 70% yield. Compound 3 was hydrolyzed and then combined with an receptor moiety (BAPTA-Me, BAPTA-F-Me, or MOBHA-Me) to synthesize Compounds 4 to 7 in yields of 65% to 89%. Compounds 4 to 7 prepared BCa-1, BCa-3ₘₑₘ, BCa−3°ₘₑₘ, and BCa-2 mito in yield of 47% to 72% through introduction of one targeting moiety of the organelles and hydrolysis of ester groups. BCa-1 and BCa-2 onto reacted with BrCH₂OC(0)Me to prepare BCa-1-AM and BCa-2ₘᵢₜₒ-AM in yield of 28% and 29%, respectively. A crude product (97% purity according to high-performance liquid chromatography) was used for additional experiments because the product was degraded slowly during the work-up (FIG. 1).

Meanwhile, FHEt-1_{lyso} was prepared from 9-methylfluorene by the method of Reaction Formula 2-1 below.

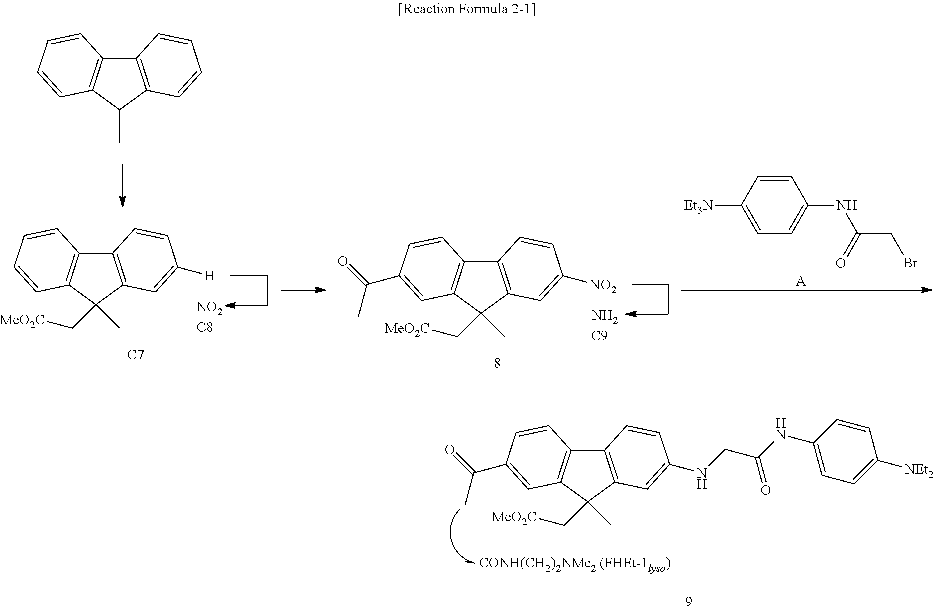

Briefly, Compound 8 was obtained in 41% yield of the total by reacting 9-methylfluorene with BuLi and then through an alkylation process using methyl bromoacetate, nitration, and acylation. Compound 9 was obtained in 46% yield through a reduction reaction of a nitro group and a reaction between an amino group and Compound A. After hydrolysis of the ester group, FHEt-1_{lyso} was prepared in yield of 40% through binding to 4-diethylaminoaniline.

Structures and specific synthetic processes of each compound of Reaction Formulas 1-1 and 1-2 were shown below.

1-1. Preparation of Compound 1

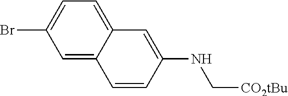 [SMILES: CC(C)(C)OC(=O)CNc1ccc2cc(Br)ccc2c1]

A solution of 6-bromo-2-aminonaphthalene (6.7 g, 30 mmol), K₂CO₃ (12.4 g, 90 mmol), and BrCH₂CO₂Bu-t (8.8 g, 45 mmol) in MeCN (60 mL) was refluxed for 12 hours. The mixture was cooled to room temperature (RT), diluted with distilled water, and then extracted with ethyl acetate. The organic layer was washed with brine, dried with anhydrous Na₂SO₄ and then the solvent was evaporated. The residue was purified through silica gel column chromatography using hexane/EtOAc (3: 1) as a mobile phase to obtain Compound 1 in a white solid state (yield 7.8 g, 78%).

¹H NMR (500 MHz, CDCl₃): δ 7.82 (1H, d, J=2.0 Hz), 7.54 (1H, d, J=8.8 Hz), 7.47 (1H, d, J=8.7 Hz), 7.42 (1H, dd, J=8.7, 2.0 Hz), 6.94 (1H, dd, J=8.8, 2.4 Hz), 6.67 (1H, d, J=2.4 Hz), 4.56 (1 H, br s), 3.89 (2 H, s), 1.51 (9 H, s) ppm; ¹³C NMR (75 MHz, CDCl₃): δ 169.9, 145.0, 133.5, 129.6, 129.5, 128.7, 128.1, 127.6, 118.8, 115.3, 104.4, 82.2, 46.3, 28.0(3) ppm.

1-2. Preparation of Compound 2

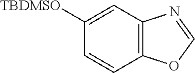 [SMILES: CC(C)(C)[Si](C)(C)Oc1ccc2ocnc2c1]

A solution containing 5-hydroxybenzoxazole (9.0 g, 67 mmol), t-butyldimethylsily chloride (TBDMS-Cl; 12.1 g, 80 mmol), imidazole (0.45 g, 6.7 mmol), and Et₃N (8.1 g, 80 mmol) in CH₂Cl₋₂ (200 mL) was stirred at room temperature for 1 hour. The mixture was added with distilled water and a product was extracted with CH₂Cl₋₂. The organic layer was washed with brine and dried with anhydrous MgSO₄ and then the solvent was evaporated. The residue was purified by silica gel column chromatography using hexane/EtOAc (3: 1) as a mobile phase to obtain Compound 2 in a pale yellow oil state (yield 15.7 g, 94%).

¹H NMR (500 MHz, CDCl₃): δ 8.03 (1 H, s), 7.39 (1H, d, J=8.8 Hz), 7.22 (1 H, d, J=2.4 Hz), 6.89 (1H, dd, J=8.8, 2.4 Hz), 0.99 (9 H, s), 0.20 (6 H, s) ppm; ¹³C NMR (75 MHz, CDCl₃): δ 153.2, 152.9, 145.0, 140.8, 118.7, 110.7, 110.7, 25.6(3), 18.2,-4.6(2) ppm.

1-3. Preparation of Compound 3

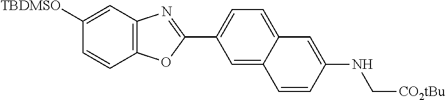 [SMILES: CC(C)(C)OC(=O)CNc1ccc2cc(-c3nc4cc(O[Si](C)(C)C(C)(C)C)ccc4o3)ccc2c1]

Compound 1 (1.1 g, 3.3 mmol), Compound 2 (1.3 g, 5.0 mmol), Cs₂CO₃ (1.3 g, 5.0 mmol), CuI (0.13 g, 0.67 mmol), Xphos (0.16 mg, 0.33 mmol), and Pd(OAc)₂ (0.036 g, 0.16 mmol) were added sequentially to DMF (10 mL) in an argon (Ar) atmosphere. The mixture was stirred at 120° C. for 3 hours, cooled to room temperature, diluted with CH₂Cl₋₂, and stirred for 10 minutes. Thereafter, the mixture was filtered through a Celite pad and washed with CH₂Cl₋₂. The filtrate was evaporated, and the residue was purified by silica gel column chromatography using hexane/EtOAc (1: 1) as a mobile phase to obtain Compound 3 in a pale yellow solid state (yield 0.90 g, 70%).

¹H NMR (500 MHz, CD₃OD/CDCl₃ at 1:4):δ 8.48 (1H, d, J=1.7 Hz), 8.04 (1H, dd, J=8.8, 1.7 Hz), 7.73 (1H, d, J=8.8 Hz), 7.66 (1H, d, J=8.8 Hz), 7.37 (1H, dd, J=8.8 Hz), 7.09 (1H, d, J=2.4 Hz), 6.99 (1 H, dt, J=8.8, 2.4 Hz), 6.83 (1 H, dd, J=8.8, 2.4 Hz), 6.69 (1H, d, J=2.4 Hz), 3.91 (2 H, s), 1.47 (9 H, s) ppm; ¹³C NMR (75 MHz, CD₃OD/CDCl₃ at 1/4):δ 170.1, 164.4, 154.3, 146.5, 144.4, 142.1, 136.6, 130.0, 127.8, 126.6, 126.4, 123.9, 119.7, 118.6, 113.2, 110.3, 104.0, 103.8, 82.3, 45.8, 27.7(3) ppm.

1-4. Preparation of Compound C1

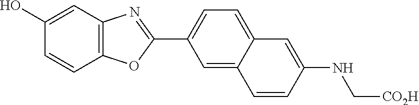 [SMILES: O=C(O)CNc1ccc2cc(-c3nc4cc(O)ccc4o3)ccc2c1]

A mixture of TFA and CH₂Cl₋₂ (1: 1, 20 mL) was dropped to a solution of Compound 3 (3.0 g, 7.7 mmol) in CH₂Cl₋₂ (10 mL) at 0° C., stirred at room temperature overnight, and added with diethyl ether (30 mL), and then the solvent was evaporated. The formed precipitate was resuspended in diethyl ether, filtered, and washed with diethyl ether. The product (Compound C1) was obtained in the form of a dark yellow solid (yield 2.3 g, 89%).

¹H NMR (500 MHz, DMSO-d₆): δ 9.51 (1 H, br s), 8.50 (1H, d, J=1.7 Hz), 8.01 (1H, dd, J=8.7, 1.7 Hz), 7.85 (1H, d, J=8.8 Hz), 7.71 (1H, d, J=8.7 Hz), 7.55 (1H, d, J=8.8 Hz), 7.15 (1H, dd, J=8.8, 2.4 Hz), 7.07 (1H, d, J=2.4 Hz), 6.81 (1H, dd, J=8.8, 2.4 Hz), 6.74 (1H, d, J=2.4 Hz), 3.97 (2 H, s) ppm; ¹³C NMR (75 MHz, DMSO-d₆): δ 172.3, 163.7, 155.0, 148.2, 144.0, 142.9, 136.7, 129.9, 127.5, 126.4, 125.9, 123.9, 119.2, 119.1, 113.2, 110.6, 104.6, 102.6, 44.5 ppm.

1-5. Preparation of Compound 4

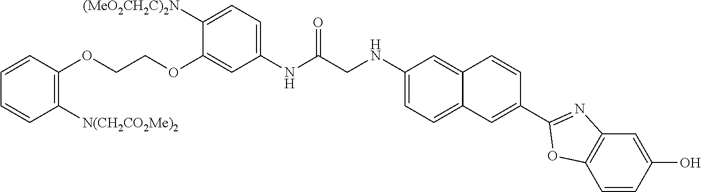

1-[bis(dimethylamino)methylene]-1H-1,2,3-triazolo[4,5-b]pyridinium 3-oxide hexafluorophosphate (HATU; 0.57 g, 1.5 mmol) was added to a solution containing Compound C1 (0.42 g, 1.3 mmol), BAPTA-Me (0.65 g, 1.2 mmol), and (i-Pr)₂NH (0.43 mL, 2.5 mmol) in DMF (3.8 mL). The mixture was stirred at room temperature for 1 hour, then added with distilled water and the product was extracted with CH₂Cl₂. The organic layer was washed with brine and dried with anhydrous Na₂SO₄, and then the solvent was evaporated. The residue was purified by silica gel column chromatography using CH₂Cl₂/EtOAc (1: 2) as a mobile phase to obtain Compound C4 in a pale yellow solid state (yield 1.0 g, 89%).

¹H NMR (500 MHz, CD₃CN): δ 8.60 (1 H, br s), 8.55 (1H, s), 8.09 (1 H, dd, J=8.8, 1.8 Hz), 7.87 (1 H, d, J=8.9 Hz), 7.75 (1H, d, J=8.8 Hz), 7.46 (1H, d, J=8.8 Hz), 7.26 (1H, d, J=2.3 Hz), 7.15 (1H, dd, J=8.9, 2.4 Hz), 7.12 (1H, d, J=2.4 Hz), 7.05 (1H, dd, J=8.7, 2.3 Hz), 7.03 (1H, s), 6.82-6.92 (5H, m), 6.74-6.79 (2H, m), 5.52 (1H, t, J=5.9 Hz), 4.16-4.21 (4H, m), 4.05 (4H, s), 4.04 (4H, s), 4.00 (2H, d, J=5.6 Hz), 3.50 (6 H, s), 3.48 (6 H, s) ppm; ¹³C NMR (75 MHz, CD₃CN): δ 172.7(2), 172.6(2), 169.4, 165.2, 155.3, 151.0, 150.8, 148.6, 145.9, 144.2, 139.9, 137.8, 136.2, 133.9, 130.9, 128.4, 127.8, 127.6, 125.2, 122.6, 122.0, 121.5, 120.2, 119.6, 119.2, 113.9, 113.8, 113.1, 111.4, 106.4, 105.7, 104.8, 68.0, 67.8, 54.0(4), 52.1(4), 48.7 ppm.

1-6. Preparation of Compound C3

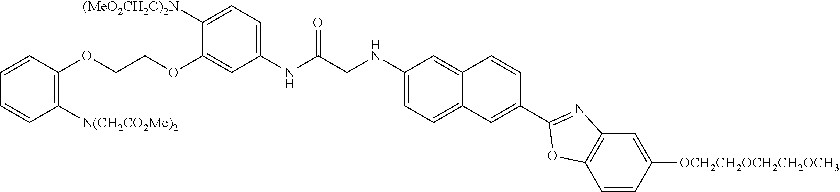

A solution of Compound 4 (0.57 g, 0.66 mmol), 2-(2-methoxyethoxy)ethyl 4-methyl benzenesulfonate (0.24 g, 0.86 mmol) and K₂CO₃ (0.14 g, 0.99 mmol) in DMF (2.2 mL) was stirred at 90° C. for 10 hours, and then the mixture was diluted with distilled water, and extracted with CH₂Cl₋₂. The organic layer was washed with brine and dried with anhydrous MgSO₄ and then the solvent was evaporated. The residue was purified by silica gel column chromatography using EtOAc as the mobile phase to obtain Compound C3 in a yellow solid state (yield 0.56 g, 84%).

¹H NMR (500 MHz, CDCl₃): δ 8.60 (1H, s), 8.35 (1H, s), 8.18 (1H, dd, J=8.7, 1.8 Hz), 7.83 (1 H, d, J=8.9 Hz), 7.73 (1 H, d, J=8.7 Hz), 7.45 (1 H, d, J=8.8 Hz), 7.29 (1H, d, J=2.0 Hz), 7.25 (1H, d, J=1.8 Hz), 7.05 (1H, dd, J=8.9, 1.8 Hz), 6.96 (2H, dd, J=8.8, 2.4 Hz), 6.81-6.92 (6H, m), 6.77 (1H, d, J=8.7 Hz), 4.24-4.29 (4H, m), 4.20--4.22 (2H, m), 4.14 (4H, s), 4.10 (4H, s), 4.05 (2 H, s), 3.90-3.93 (2H, m), 3.74-3.77 (2H, m), 3.60-3.62 (2H, m), 3.57 (6H, s), 3.54 (6H, s), 3.41 (3 H, s) ppm; ¹³C NMR (75 MHz, CDCl₃): δ 171.9(2), 171.8(2), 168.2, 164.2, 156.1, 150.3, 150.1, 146.3, 145.2, 142.7, 139.0, 136.3, 135.6, 132.4, 130.2, 127.6, 127.1, 126.7, 124.4, 122.2, 121.3, 120.8, 118.9, 118.8, 118.4, 113.6, 112.7, 112.4, 110.3, 105.5, 105.0, 103.1, 71.8, 70.6, 69.7, 67.9, 67.0, 66.7, 59.0, 53.2(4), 51.6(4), 48.8 ppm.

1-7. Preparation of BCa-1

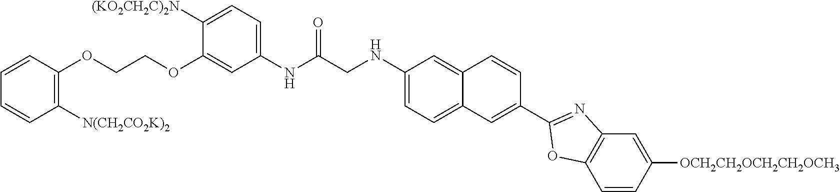

A solution of Compound C3 (0.024 g, 0.025 mmol) in dioxane (0.20 mL) and EtOH (0.30 mL) was dropped with KOH (aq) (1 N, 0.20 mL, 0.20 mmol) while stirring at 0° C. The mixture was stirred at room temperature for 5 hours, and then dropped with HCl (aq) (1 N, 0.10 mL, 0.10 mmol). After 10 minutes, the mixture was dropped to a vial containing MeCN (2 mL) while stirring. The formed precipitate was obtained by filtration and washed with diethyl ether to obtain BCa-1 in a yellow solid state (yield 0.018 g, 67%).

¹H NMR (500 MHz, DMSO-d₆): δ 8.46 (1H, s), 7.96 (1H, d, J=8.7 Hz), 7.82 (1H, d, J=8.9 Hz), 7.72 (1H, d, J=8.7 Hz), 7.55 (1H, d, J=8.7 Hz), 7.20 (1H, d, J=2.3 Hz), 7.09 (1H, d, J=8.9 Hz), 6.99 (1 H, br s), 6.95 (2H, dd, J=9.0, 2.3 Hz), 6.76-6.87 (7H, m), 4.15-4.23 (4H, m), 4.08-4.12 (2H, m), 3.99 (2 H, s), 3.72-3.75 (2H, m), 3.56-3.59 (2H, m), 3.44-3.47 (2H, m), 3.42 (4H, s), 3.39 (4H, s), 3.21 (3 H, s) ppm; ¹³C NMR (151 MHz, DMSO-d₆): δ 178.0(2), 177.9(2), 171.7, 165.6, 157.4, 151.3, 151.3, 149.1, 146.1, 143.0, 141.5, 138.6, 138.0, 133.7, 131.6, 129.1, 128.1, 127.6, 125.2, 123.9, 122.4, 120.5, 120.1, 119.6(2), 115.3, 114.7, 113.2, 112.6, 106.8, 104.7, 104.3, 72.3, 70.8, 70.2, 69.2, 67.4, 67.1, 59.4(4), 58.8(2) ppm. HRMS (electrospray ionization; ESI) m/z [M+K]⁺ calculated for C₄₆H₄₇KN₅O₁₅: 948.2701, found: 948.2706.

1-8. Preparation of BCa-1-AM

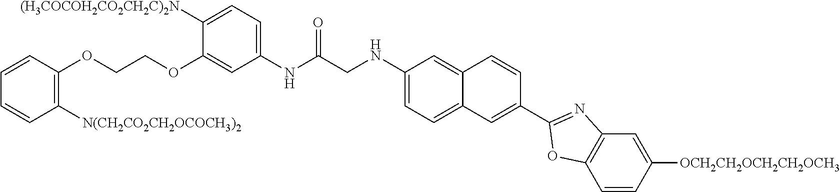

A solution of BCa-1 (0.045 g, 0.049 mmol), bromomethyl acetate (0.075 g, 0.49 mmol), and DIPEA (0.050 g, 0.39 mmol) in MeCN (0.99 mL) was stirred at room temperature for 40 hours under an Ar atmosphere. Thereafter, the product was extracted with CH₂Cl₋₂, washed with brine, and dried with anhydrous Na₂SO₄ and then the solvent was evaporated. The residue was purified by semi-preparative reverse-phase high-performance liquid chromatography (RP-HPLC; YMC-PACK ODS-A, 5 μm, 250×20 mm). At this time, RP-HPLC was performed on a YMC HPLC system (YMC LC-Forte/R 100) using MeCN/H₂O (1: 1) containing 0.1% formic acid at a flow rate of 10 mL/min for 20 minutes. The obtained product, BCa-1AM was a yellow solid, and the yield was 0.016 g (28%).

¹H NMR (500 MHz, CDCl₃): δ 8.59 (1H, s), 8.39 (1H, s), 8.17 (1H, d, J=9.0 Hz), 7.82 (1H, d, J=9.0 Hz), 7.73 (1H, d, J=9.0 Hz), 7.44 (1H, d, J=9.0 Hz), 7.31 (1H, d, J=2.0 Hz), 7.25 (1H, d, J=2.1 Hz), 7.04 (1 H, dd, J=9.0, 2.1 Hz), 7.00 (1H, d, J=8.5 Hz), 6.96 (1H, d, J=8.7 Hz), 6.93 (1H, d, J=8.5 Hz), 6.84-6.90 (4H, m), 6.82 (1H, d, J=8.7 Hz), 5.64 (4H, s), 5.60 (4H, s), 4.82 (1H, t, J=5.4 Hz), 4.25-4.32 (4H, m), 4.19-4.22 (2H, m), 4.18 (4H, s), 4.15 (4H, s), 4.05 (2H, d, J=5.2 Hz), 3.89-3.91 (2H, m), 3.74-3.77 (2H, m), 3.59-3.62 (2H, m), 3.41 (3H, s), 2.05 (12 H, s) ppm; ¹³C NMR (151 MHz, CDCl₃): δ 170.2(2), 170.0(2), 169.6(2), 169.5(2), 168.0, 164.3, 156.4, 150.8, 150.5, 146.2, 145.5, 143.0, 138.6, 136.4, 135.4, 132.9, 130.5, 127.8, 127.6, 126.9, 124.8, 123.0, 121.7, 121.5, 120.3, 119.8, 118.4, 113.9, 113.6, 112.9, 110.5, 106.4, 105.6, 103.7, 79.3(2), 79.2(2), 71.9, 70.8, 69.8, 68.2, 67.4, 67.0, 59.1, 53.4(2), 53.4, 53.3, 49.2, 20.7(2), 20.7(2) ppm. HRMS (ESI) m/z [M+Na]⁺ calcd for C₅₈H₆₃N₅NaO₂₃: 1220.3806, found: 1220.3807.

1-9. Preparation of Compound 5

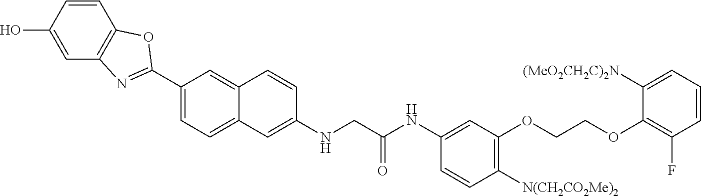

HATU (0.55 g, 1.4 mmol) was added to a solution of Compound C1 (0.40 g, 1.2 mmol), BAPTA-F-Me (0.68 g, 1.2 mmol), and (i-Pr)₂NH (0.41 mL, 2.4 mmol) in DMF (4 mL). The mixture was stirred at room temperature for 2 hours, then added with distilled water and the product was extracted with CH₂Cl₂. The organic layer was washed with brine and dried with anhydrous Na₂SO₄ and then the solvent was evaporated. The residue was purified through silica gel column chromatography using CH₂Cl₂/EtOAc (1: 2) as a mobile phase to obtain Compound 5 (0.68 g, 65%) in a pale yellow solid state.

¹H NMR (500 MHz, CDCl₃): δ 8.60 (d, J=1.8 Hz, 1H), 8.18 (dd, J=8.8, 1.8 Hz, 1H), 8.18 (br s, 1H), 7.81 (d, J=8.8 Hz, 1H), 7.71 (d, J=8.7 Hz, 1H), 7.41 (d, J=8.7 Hz, 1H), 7.26 (br s, 1H), 7.20 (d, J=2.5 Hz, 1H), 7.05-6.97 (m, 2H), 6.92-6.83 (m, 4H), 6.72-6.65 (m, 2H), 4.39-4.35 (m, 2H), 4.30-4.26 (m, 2H), 4.17 (s, 4H), 4.14 (s, 4H), 4.03 (s, 2H), 3.66 (s, 6H), 3.63 (s, 6H) ppm; ¹³C NMR (75 MHz, CD₃OD/CDCl₃ at 1/9): δ 171.9(2), 171.5(2), 168.4, 164.2, 156.4 (d, J=245.1 Hz), 154.4, 150.6, 146.6, 144.6, 143.7 (d, J=3.8 Hz), 142.3, 138.3 (d, J=12.8 Hz), 136.5, 135.6, 132.3, 130.3, 127.8, 127.2, 126.8, 124.2, 123.6 (d, J=9.7 Hz), 120.6, 119.8, 118.5, 114.7, 113.4, 112.5, 110.4, 109.9 (J=19.8 Hz), 106.1, 104.9, 104.3, 71.3, 67.6, 53.4(4), 51.7(2), 51.6(2), 48.4 ppm.

1-10. Preparation of Compound C4

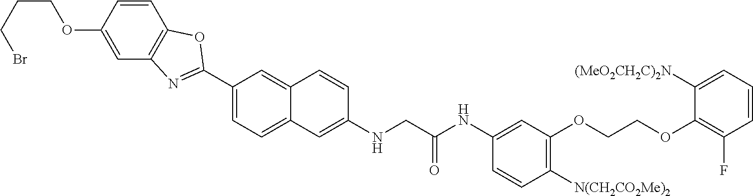

A solution of Compound 5 (0.55 g, 0.62 mmol), 1,3-dibromopropane (0.31 mL, 3.1 mmol), and K₂CO₃ (0.34 g, 2.5 mmol) in DMF (2.5 mL) was stirred for 6 hours at 72° C. Then, distilled water was added and the product was extracted using CH₂Cl₂. The organic layer was washed with brine and dried with anhydrous Na₂SO₄ and then the solvent was evaporated. The residue was purified through silica gel column chromatography using EtOAc as a mobile phase to obtain Compound C4 (0.37 g, 60%) in a pale yellow solid state.

¹H NMR (500 MHz, CDCl₃): δ 8.58 (d, J=1.8 Hz, 1H), 8.35 (s, 1H), 8.17 (dd, J=8.8, 1.8 Hz, 1H), 7.80 (d, J=8.8 Hz, 1H), 7.71 (d, J=8.8 Hz, 1H), 7.46 (d, J=8.8 Hz, 1H), 7.26 (br s, 1H), 7.26 (s, 1H), 7.02 (dd, J=8.8, 2.4 Hz, 1H), 7.00-6.81 (m, 5H), 6.71-6.62 (m, 2H), 4.38-4.34 (m, 2H), 4.25-4.21 (m, 2H), 4.18-4.14 (m, 2H), 4.17 (s, 4H), 4.16 (s, 4H), 4.02 (s, 2H), 3.65 (s, 6H), 3.64 (t, J=6.1 Hz, 2H), 3.61 (s, 6H), 2.36 (p, J=6.1 Hz, 2H) ppm; ¹³C NMR (75 MHz, CDCl₃): δ 171.7(2), 171.3(2), 167.9, 164.4, 156.5 (d, J=245.4 Hz), 156.3, 150.7, 146.4, 145.5, 143.8 (d, J=3.7 Hz), 143.0, 138.4 (d, J=12.6 Hz), 136.4, 135.6, 132.5, 130.4, 127.8, 127.4, 126.9, 124.6, 123.7 (d, J=9.4 Hz), 121.2, 119.8, 118.4, 114.8, 113.6, 112.5, 110.5, 110.0 (d, J=19.8 Hz), 106.2, 105.4, 103.6, 71.3, 67.7, 66.1, 53.4(4), 51.7(2), 51.6(2), 49.0, 32.3, 30.0 ppm.

1-11. Preparation of Compound C5

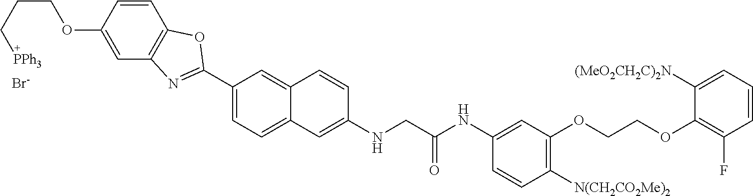

A solution of Compound C4 (0.21 g, 0.21 mmol) and Ph₃P (0.55 g, 2.1 mmol) in MeCN (2 mL) was stirred at 90° C. for 12 hours. The solvent was evaporated and the residue was purified through silica gel column chromatography using CH₂Cl₂/MeOH (10: 1) as a mobile phase to obtain Compound C5 (0.11 g, 40%) in a yellow solid state.

¹H NMR (500 MHz, CDCl₃): δ 9.46 (br s, 1H), 8.16 (d, J=1.7 Hz, 1H), 7.80 (dd, J=8.9, 1.7 Hz, 1H), 7.77-7.66 (m, 9H), 7.67-7.58 (m, 6H), 7.47 (d, J=2.3 Hz, 1H), 7.44 (d, J=8.9 Hz, 1H), 7.37 (d, J=8.7 Hz, 1H), 7.15 (dd, J=8.7, 2.2 Hz, 1H), 7.12 (d, J=8.8 Hz, 1H), 7.08 (dd, J=8.7, 2.3 Hz, 1H), 6.91-6.81 (m, 2H), 6.72 (d, J=8.7 Hz, 1H), 6.69 (dd, J=8.8, 2.4 Hz, 1H), 6.66-6.60 (m, 3H), 6.23 (s, 1H), 4.34-4.29 (m, 2H), 4.21-4.11 (m, 8H), 4.11 (s, 4H), 4.01 (s, 2H), 3.86-3.76 (m, 2H), 3.60 (s, 6H), 3.59 (s, 6H), 2.08-2.02 (m, 2H) ppm; ¹³C NMR (75 MHz, CDCl₃): δ 171.8(2), 171.3(2), 169.0, 164.2, 156.5 (d, J=245.5 Hz), 155.3, 150.5, 147.1, 145.2, 143.8 (d, J=3.9 Hz), 142.7, 138.4 (d, J=12.9 Hz), 136.4, 135.2(3) (d, J=3.0 Hz), 134.9, 133.6, 133.4(6) (d, J=10.0 Hz), 130.5 (6) (d, J=12.6 Hz), 129.7, 127.5, 126.6, 126.4, 123.9, 123.6 (d, J=9.3 Hz), 119.8, 119.7, 119.0, 117.7 (3) (d, J=86.4 Hz), 114.9, 112.4, 112.2, 110.2, 109.9 (d, J=19.4 Hz), 105.8, 104.3, 103.6, 71.4, 67.5, 66.9 (d, J=16.1 Hz), 53.4(4), 51.7(2), 51.5(2), 48.6, 22.8, 19.6 (d, J=53.4 Hz) ppm.

1-12. Preparation of BCa-2ₘᵢₜₒ

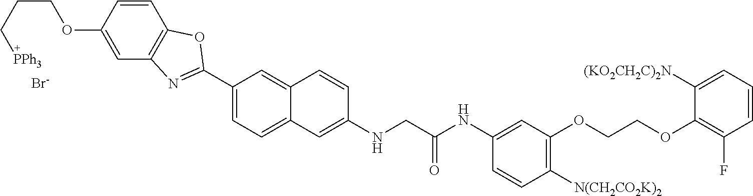

A solution of Compound C5 (0.022 g, 0.017 mmol) in dioxane (0.070 mL) and MeOH (0.21 mL) was dropped with KOH (aq) (1 N, 0.14 mL, 0.14 mmol) while stirring at 0° C. The mixture was stirred at room temperature for 24 hours, and then dropped with HCl (aq) (1 N, 0.070 mL, 0.070 mmol). The crude product (97% purity according to HPLC) was used in the next step without additional purification.

HRMS (ESI) m/z [M]⁺ calcd for C₆₂H₅₆FN₅O₁₃P⁺: 1128.3591, found: 1128.3587.

1-13. Preparation of BCa-2ₘᵢₜₒ-AM

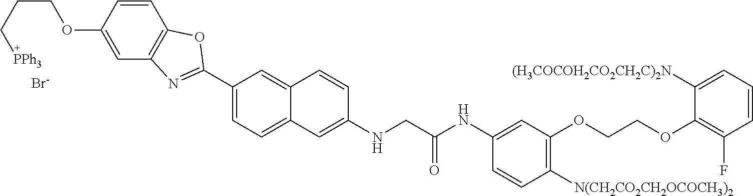

A solution of BCa-2 mite (0.089 g, 0.074 mmol), bromomethyl acetate (0.28 g, 1.9 mmol), and DIPEA (0.19 g, 1.5 mmol) in MeCN (1.4 mL) and DMF (0.90 mL) was stirred at room temperature for 72 hours under an Ar atmosphere. The product was extracted with CH₂Cl₋₂, washed with brine, and dried with anhydrous Na₂SO₄ and then the solvent was evaporated. The residue was purified by performing medium-pressure liquid chromatography (Yamazen ODS-SM, 50 μm, 2.3×12.3 cm) in a Yamazen MPLC system (Yamazen W-Prep 2XY) using MeCN/H₂O (1: 1) containing 0.1% formic acid as a mobile phase at a flow rate of 10 mL/min for 20 minutes. The obtained product, BCa-2ₘᵢₜₒ-AM was a pale yellow solid, and the yield was 0.032 g (29%).

¹H NMR (500 MHz, CD₃CN): δ9.15 (br s, 1H), 8.50 (d, J=1.8 Hz, 1H), 8.05 (dd, J=8.7, 1.8 Hz, 1H), 7.88-7.81 (m, 4H), 7.77-7.67 (m, 13H), 7.51 (d, J=8.9 Hz, 1H), 7.34 (d, J=2.2 Hz, 1H), 7.21 (d, J=2.4 Hz, 1H), 7.17 (dd, J=8.9, 2.2 Hz, 1H), 7.12 (dd, J=8.5, 2.4 Hz, 1H), 6.97-6.91 (m, 2H), 6.86 (d, J=2.0 Hz, 1H), 6.80 (d, J=8.5 Hz, 1H), 6.77-6.70 (m, 2H), 5.89 (t, J=5.6 Hz, 1H), 5.62 (s, 4H), 5.60 (s, 4H), 4.33-4.29 (m, 2H), 4.22-4.11 (m, 12H), 4.02 (d, J=5.6 Hz, 2H), 3.49-3.42 (m, 2H), 2.13-2.06 (m, 2H), 1.97 (s, 6H), 1.96 (s, 6H) ppm; ¹³C NMR (75 MHz, CDCl₃): δ170.0(2), 169.5(2), 169.4(2), 169.0(2), 166.9, 164.4, 156.6 (d, J=245.3 Hz), 155.2, 150.8, 147.2, 145.3, 143.3 (d, J=3.3 Hz), 142.9, 138.7 (d, J=12.7 Hz), 136.6, 135.3(3) (d, J=2.8 Hz), 134.3, 134.2, 133.2(6) (d, J=9.9 Hz), 130.5(6) (d, J=12.5 Hz), 129.8, 127.6, 126.5, 126.4, 124.0, 123.7 (d, J=9.4 Hz), 120.4, 119.8, 118.9, 117.5(3) (d, J=86.5 Hz), 115.3, 115.3, 112.4, 110.4 (d, J=19.3 Hz), 110.3, 106.0, 104.1, 103.6, 79.0(4), 71.4, 67.6, 66.7 (d, J=16.3 Hz), 53.3(4), 48.4, 22.6, 20.6(4), 19.3 (d, J=53.8 Hz) ppm. HRMS (ESI) m/z [M]⁺ calcd for C₇₄H₇₂PN₅O₂₁P⁺: 1416.4436, found: 1416.4442.

1-14. Preparation of Compound 6

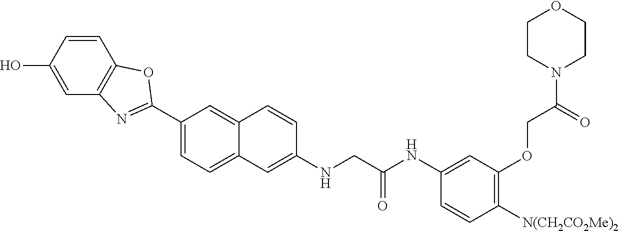

HATU (0.89 g, 2.3 mmol) was added to a solution of Compound C1 (0.68 g, 2.0 mmol), MOBHA-Me (0.76 g, 1.9 mmol), and (i-Pr)₂NH (0.69 mL, 2.0 mmol) in DMF (6 mL), and the mixture was stirred at room temperature for 30 minutes. The mixture was added with distilled water and extracted with EtOAc, and then the organic layer was washed with brine and dried with anhydrous Na₂SO₄ and the solvent was evaporated. The product was purified through silica gel column chromatography using EtOAc/i-PrOH (98: 2) as a mobile phase to obtain Compound 6 in a pale yellow solid state (yield 1.09 g, 78%).

¹H NMR (500 MHz, DMSO-d₆/CD₃CN at 1:5):δ 9.36 (s, 1H), 9.08 (s, 1H), 8.56 (d, J=1.7 Hz, 1H), 8.10 (dd, J=8.8, 1.7 Hz, 1H), 7.88 (d, J=8.8 Hz, 1H), 7.76 (d, J=8.8 Hz, 1H), 7.48 (d, J=8.6 Hz, 1H), 7.26 (d, J=2.3 Hz, 1H), 7.20 (dd, J=8.8, 2.4 Hz, 1H), 7.13 (d, J=2.4 Hz, 1H), 7.09 (dd, J=8.6, 2.3 Hz, 1H), 6.88 (dd, J=8.6, 2.4 Hz, 1H), 6.86 (d, J=2.4 Hz, 1H), 6.83 (d, J=8.6 Hz, 1H), 6.08 (t, J=5.9 Hz, 1H), 4.77 (s, 2H), 4.15 (s, 4H), 4.03 (d, J=5.9 Hz, 2H), 3.66-3.58 (m, 4H), 3.65 (s, 6H), 3.52-3.44 (m, 4H) ppm; ¹³C NMR (75 MHz, DMSO-d₆/CD₃CN at 1:5):δ 171.5(2), 168.5, 166.0, 164.0, 155.2, 149.6, 148.2, 144.4, 143.2, 136.9, 134.8, 133.6, 130.0, 127.5, 126.5, 126.5, 124.2, 120.0(2), 119.4, 113.2, 112.6, 110.4, 105.9, 104.7, 103.3, 66.3, 66.2, 66.0, 53.1(2), 51.3(2), 47.3, 45.0, 41.8 ppm.

1-15. Preparation of Compound C6

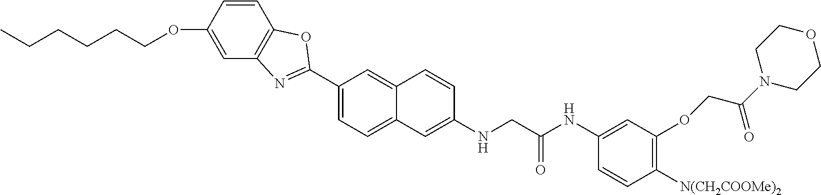

A solution of Compound 6 (0.15 g, 0.21 mmol), 1,3-dibromopropane (31 μL, 0.23 mmol), and K₂CO₃ (37 mg, 0.27 mmol) dissolved in DMF (0.7 mL) was stirred at 80° C. for 16 hours. Next, distilled water was added and the product was extracted with EtOAc, and then the organic layer was washed with brine and dried with anhydrous Na₂SO₄ and the solvent was evaporated. The residue was purified through silica gel column chromatography using CH₂Cl₂/EtOAc (1: 10) as a mobile phase to obtain Compound C6 in a white solid state (yield 0.14 g, 85%).

¹H NMR (500 MHz, CDCl₃): δ 8.61 (d, J=1.7 Hz, 1H), 8.35 (br s, 1H), 8.19 (dd, J=8.7, 1.7 Hz, 1H), 7.83 (d, J=8.7 Hz, 1H), 7.73 (d, J=8.7 Hz, 1H), 7.45 (d, J=8.7 Hz, 1H), 7.35 (d, J=2.3 Hz, 1H), 7.25 (d, J=2.5 Hz, 1H), 7.05 (dd, J=8.7, 2.3 Hz, 1H), 6.95 (dd, J=8.7, 2.3 Hz, 1H), 6.94 (dd, J=8.9, 2.5 Hz, 1H), 6.87 (d, J=8.9 Hz, 1H), 6.87 (d, J=2.3 Hz, 1H), 4.74 (s, 2H), 4.12 (s, 4H), 4.05-3.99 (m, 4H), 3.67-3.64 (m, 4H), 3.66 (s, 6H), 3.62-3.58 (m, 2H), 3.53-3.49 (m, 2H), 1.82 (m, 2H), 1.49 (m, 2H), 1.36 (m, 4H), 0.95-0.87 (m, 3H) ppm; ¹³C NMR (75 MHz, CDCl₃): δ 171.5(2), 168.1, 166.2, 164.2, 156.8, 150.0, 146.3, 145.2, 143.0, 136.4, 135.7, 132.8, 130.4, 127.7, 127.4, 126.8, 124.6, 121.3, 120.3, 118.4, 113.8, 113.6, 110.4, 107.2, 105.4, 103.4, 68.8, 67.3, 66.7, 66.6, 53.5(2), 51.7(2), 49.0, 45.4, 42.2, 31.6, 29.2, 25.7, 22.6, 14.0 ppm.

1-1.6 Preparation of BCa-3ₘₑₘ

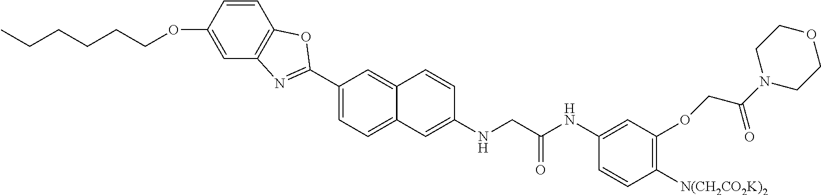

KOH (aq) (1 N, 0.087 mL, 0.087 mmol) was dropped to a solution of Compound C6 (0.034 g, 0.043 mmol) in dioxane (0.20 mL) and EtOH (0.52 mL) while stirring at 0° C. After stirring at room temperature for 48 hours, the mixture was dropped to a vial containing MeCN (2.5 mL). The formed precipitate was collected by filtration and washed with diethyl ether. The obtained product, BCa-3ₘₑₘ was a yellow solid and the yield was 0.024 g (72%).

¹H NMR (500 MHz, DMSO-d₆/CD₃CN at 9:1):δ 10.51 (s, 1H), 8.50 (d, J=1.8 Hz, 1H), 8.01 (dd, J=8.8, 1.8 Hz, 1H), 7.84 (d, J=8.8 Hz, 1H), 7.70 (d, J=8.8 Hz, 1H), 7.62 (d, J=8.8 Hz, 1H), 7.39 (s, 1H), 7.30 (d, J=2.5 Hz, 1H), 7.19 (dd, J=8.8, 2.3 Hz, 1H), 7.09 (br s, 1H), 6.95 (dd, J=8.8, 2.5 Hz, 1H), 6.85 (d, J=8.8 Hz, 1H), 6.79 (s, 1H), 4.79 (s, 2H), 4.05-3.98 (m, 4H), 3.65-3.61 (m, 2H), 3.57-3.53 (m, 2H), 3.48-3.42 (m, 4H), 3.21 (br s, 4H), 1.78-1.71 (m, 2H), 1.49-1.42 (m, 2H), 1.36-1.30 (m, 4H), 0.92-0.87 (m, 3H) ppm; ¹³C NMR (75 MHz, DMSO-d₆): δ174.2(2), 168.7, 166.9, 164.2, 156.8, 150.0, 148.6, 145.0, 143.0, 137.3, 137.1, 133.5, 130.3, 127.9, 126.9, 126.3, 124.3, 119.6, 119.2, 119.0, 113.8, 112.2, 111.3, 104.6, 103.6, 103.1, 68.6, 66.3, 66.2, 64.9, 59.1(2), 47.0, 44.8, 42.0, 31.4, 29.0, 25.6, 22.5, 14.4 ppm. HRMS (ESI) m/z [M+K]⁺ calcd for C₄₁H₄₅KN₅O₁₀: 806.2798, found: 806.2800.

1-17. Preparation of Compound 7

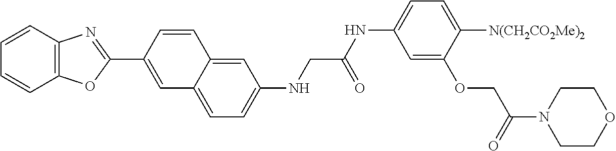

HATU (0.99 g, 2.6 mmol) was added to a solution of Compound C2 (0.76 g, 2.4 mmol), MOBHA-Me (0.89 g, 2.3 mmol), and (i-Pr)₂NH (0.81 mL, 4.7 mmol) in DMF (7.5 mL), and the mixture was stirred at room temperature for 30 minutes. Distilled water was then added and the product was extracted with EtOAc. The organic layer was washed with brine and dried with anhydrous Na₂SO₄ and then the solvent was evaporated. The residue was purified through silica gel column chromatography using CH₂Cl₂/EtOAc (1: 3) as a mobile phase to obtain Compound 7 in a pale yellow solid state (yield 1.17 g, 70%).

¹H NMR (500 MHz, CDCl₃): δ 8.59 (s, 1H), 8.50 (br s, 1H), 8.17 (d, J=8.7, 1H), 7.81-7.73 (m, 2H), 7.70 (d, J=8.7 Hz, 1H), 7.61-7.54 (m, 1H), 7.38-7.31 (m, 3H), 7.03 (br d, J=8.6 Hz, 1H), 6.98 (br d, J=8.6 Hz, 1H), 6.88-6.81 (m, 2H), 4.71 (s, 2H), 4.11 (s, 4H), 4.00 (s, 2H), 3.66 (s, 6H), 3.66-3.62 (m, 4H), 3.60-3.55 (m, 2H), 3.50-3.45 (m, 2H) ppm; ¹³C NMR (75 MHz, CDCl₃): δ 171.5(2), 168.2, 166.3, 163.5, 150.7, 150.0, 146.4, 142.2, 136.5, 135.7, 132.9, 130.4, 127.9, 127.4, 126.9, 124.8, 124.7, 124.4 121.1, 120.4, 119.6, 118.5, 113.6, 110.4, 107.2, 105.3, 67.3, 66.6, 66.5, 53.5(2), 51.7(2), 48.9, 45.4, 42.2 ppm.

1-18. Preparation of BCa−3°ₘₑₘ

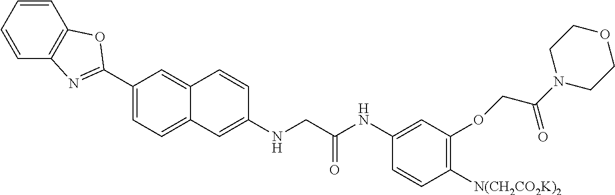

KOH (aq) (1 N, 0.18 mL, 0.18 mmol) was dropped to a solution of Compound 7 (0.050 g, 0.072 mmol) in dioxane (0.50 mL) and MeOH (0.50 mL) while stirring at 15° C., and then the mixture was stirred at room temperature for 15 hours. The reaction mixture was dropped to a round bottom flask containing MeCN (3 mL) while stirring. The formed precipitate was collected by filtration and washed with diethyl ether. The obtained product, BCa−3°ₘₑₘ was a yellow solid and the yield was 0.024 g (47%).

¹H NMR (500 MHz, DMSO-d₆): δ 8.52 (d, J=1.7 Hz, 1H), 8.02 (dd, J=8.8, 1.7 Hz, 1H), 7.84 (d, J=8.9 Hz, 1H), 7.76-7.72 (m, 2H), 7.70 (d, J=8.8 Hz, 1H), 7.40-7.36 (m, 2H), 7.27 (d, J=2.2 Hz, 1H), 7.16 (dd, J=8.9, 2.2 Hz, 1H), 6.95 (dd, J=8.7, 2.0 Hz, 1H), 6.86 (d, J=8.7 Hz, 1H), 6.76 (d, J=2.0 Hz, 1H), 4.78 (br s, 2H), 3.99 (br s, 2H), 3.61-3.55 (m, 2H), 3.53-3.47 (m, 2H), 3.44-3.37 (m, 4H), 3.34-3.26 (m, 4H) ppm; ¹³C NMR (151 MHz, DMSO-d₆): δ 174.3(2), 168.7, 166.9, 163.5, 150.5, 150.1, 148.6, 142.0, 137.2, 137.1, 133.6, 130.2, 128.0, 126.8, 126.2, 125.4, 125.2, 124.3, 119.7, 119.6, 119.1, 119.0, 112.2, 111.0, 104.6, 103.1, 66.2, 66.1, 64.8, 59.2(2), 47.0, 44.7, 42.0 ppm. HRMS (ESI) m/z [M+K]⁺ calcd for C35H₃₃KN₅O₉⁺: 706.1910, found: 706.1928.

1-19. Preparation of Compound C7

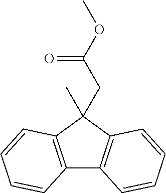 [SMILES: COC(=O)CC1(C)c2ccccc2-c2ccccc21]

t-BuONa (5.3 g, 55 mmol) was added to a solution of 9-methyl-9H-fluorene (4.0 g, 22 mmol) in DMSO (100 mL) while stirring at 10° C. Next, the solution was stirred at room temperature for 2 hours, and added with distilled water. The product was extracted with EtOAc, washed with brine, dried with anhydrous Na₂SO₄ and evaporated.

The residue was purified through silica gel column chromatography using hexane/EtOAc (5: 1) as a mobile phase to obtain Compound C7 in a white solid state (yield 3.6 g, 65%).

¹H NMR (500 MHz, CDCl₃): δ 7.77-7.74 (2H, m), 7.50-7.47 (2H, m), 7.38 (2H, td, J=7.3, 1.2 Hz), 7.34 (2H, td, J=7.3, 1.2 Hz), 3.48 (3H, s), 2.88 (2H, s), 1.63 (3H, s) ppm; ¹³C NMR (75 MHz, CDCl₃): δ 171.1, 150.7(2), 139.5(2), 127.4(2), 127.2(2), 122.9(2), 119.9(2), 51.1, 48.3, 43.9, 25.3 ppm.

1-20. Preparation of Compound C8

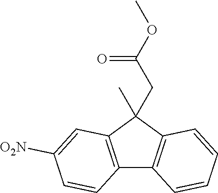 [SMILES: COC(=O)CC1(C)c2ccccc2-c2ccc([N+](=O)[O-])cc21]

HNO₃ (1.4 mL, 33 mmol) was dropped to a solution of Compound C7 (2.4 g, 8.1 mmol) in CH₂Cl₂ (16 mL) while stirring at 0° C. After stirring at 0° C. for 20 minutes, the reaction mixture was poured into a beaker containing ice and a saturated NaHCO₃(aq) solution. The product was extracted with CH₂Cl₂, washed with brine, dried with anhydrous MgSO₄ and then the solvent was evaporated. The residue was purified through silica gel column chromatography using hexane/EtOAc (4: 1) as a mobile phase to obtain Compound C8 in a white solid state (yield 1.8 g, 75%).

¹H NMR (500 MHz, CDCl₃): δ 8.35 (1H, d, J=2.2 Hz), 8.28 (1H, dd, J=8.3, 2.2 Hz), 7.83 (1H, d, J=8.3 Hz), 7.82 (1H, dd, 8.3, 0.7 Hz), 7.53-7.50 (1H, m), 7.46-7.41 (2H, m), 3.43 (3H, s), 3.02 (1H, d, J=15.2 Hz), 2.94 (1H, d, J=15.2 Hz), 1.62 (3H, s) ppm; ¹³C NMR (75 MHz, CDCl₃): δ 170.3, 152.1, 151.7, 146.9, 146.2, 137.2, 129.4, 128.0, 123.7, 123.1, 121.4, 120.1, 118.6, 51.4, 48.5, 43.2, 25.5 ppm.

1-21. Preparation of Compound 8

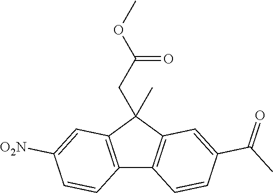 [SMILES: COC(=O)CC1(C)c2cc(C(C)=O)ccc2-c2ccc([N+](=O)[O-])cc21]

A solution of Compound C8 (3.3 g, 11 mmol) and acetyl chloride (3.8 mL, 54 mmol) in nitrobenzene (15 mL) was stirred at 40° C. for 1 hour. AlCl₃ (7.2 g, 54 mmol) was added slowly and the mixture was stirred at 60° C. for 5 hours. The mixture was cooled on an ice bath and the reaction was quenched using 1 N HCl (aq). The product was extracted with CH₂Cl₂, washed with brine, dried with anhydrous MgSO₄ and then the solvent was evaporated. The residue was purified through silica gel column chromatography using hexane/EtOAc (3: 1) as a mobile phase to obtain Compound 8 in a pale yellow solid state (yield 3.2 g, 85%).

¹H NMR (500 MHz, CDCl₃): δ 8.35 (1H, d, J=2.0 Hz), 8.30 (1H, dd, J=8.4, 2.0 Hz), 8.11 (1H, d, 1.6 Hz), 8.04 (1H, dd, J=7.9, 1.6 Hz), 7.90 (1H, d, J=6.6 Hz), 7.89 (1H, d, J=6.6 Hz), 3.40 (3H, s), 3.09 (1H, d, J=15.6 Hz), 3.04 (1H, d, J=15.6 Hz), 2.67 (3H, s), 1.63 (3H, s) ppm; ¹³C NMR (75 MHz, CDCl₃): δ 197.6, 170.0, 153.0, 152.6, 147.9, 144.9, 142.0, 137.6, 128.9, 123.9, 122.7, 121.4, 121.2, 118.7, 51.6, 48.8, 43.0, 26.9, 25.8 ppm.

1-22. Preparation of Compound C9

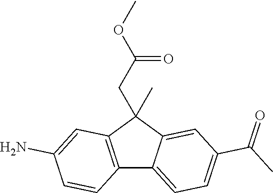 [SMILES: COC(=O)CC1(C)c2cc(N)ccc2-c2ccc(C(C)=O)cc21]

A solution of Compound 8 (2.2 g, 6.4 mmol), Fe powder (1.1 g, 19 mmol), and NH₄C1 (0.69 g, 13 mmol) in EtOH (10 mL) and 1,4-dioxane (10 mL) was refluxed for 2 hours. The mixture was cooled, neutralized by adding Na₂CO₃ (aq) and then filtered through Celite. The product was extracted with CH₂Cl₂, washed with brine, dried with anhydrous MgSO₄ and then the solvent was evaporated. The residue was purified through silica gel column chromatography using hexane/EtOAc (1: 1) as a mobile phase to obtain Compound C9 in a pale yellow solid state (yield 1.7 g, 80%).

¹H NMR (500 MHz, CDCl₃): δ 7.99 (1H, d, J=1.5 Hz), 7.93 (1H, dd, J=8.0, 1.5 Hz), 7.61 (1H, d, J=8.0 Hz), 7.57 (1H, d, J=8.1 Hz), 6.82 (1H, d, J=2.1 Hz), 6.72 (1H, dd, J=8.1, 2.1 Hz), 3.46 (3H, s), 2.89 (1H, d, J=15.0 Hz), 2.84 (1H, d, J=15.0 Hz), 2.63 (3H, s), 1.56 (3H, s) ppm; ¹³C NMR (75 MHz, CDCl₃): δ 197.7, 170.8, 154.1, 150.1, 147.6, 145.2, 134.2, 128.9, 128.8, 122.4, 122.0, 118.1, 114.5, 109.4, 51.2, 47.9, 43.6, 26.6, 25.6 ppm.

1-23. Preparation of Compound 9

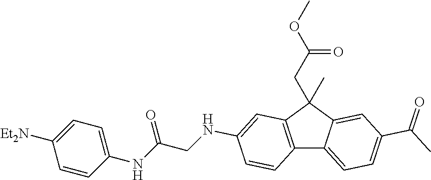

A solution of Compound C9 (3.0 g, 8.8 mmol), 2-bromo-N-(4-(diethylamino)phenyl)acetamide (5.1 g, 18 mmol), and (i-Pr)₂NH (3.1 mL, 18 mmol) in DMF (20 mL) was stirred at 80° C. for 12 hours. Distilled water was added to the mixture and the product was extracted with EtOAc. The organic layer was washed with brine and dried with anhydrous MgSO₄ and then the solvent was evaporated. The residue was purified through silica gel column chromatography using hexane/EtOAc (1:2) as a mobile phase to obtain Compound 9 in a pale yellow solid state (yield 2.6 g, 58%).

¹H NMR (500 MHz, CDCl₃): δ 8.20 (1H, s), 8.00 (1H, d, J=1.6 Hz), 7.94 (1H, dd, J=7.8, 1.6 Hz), 7.63 (1H, d, J=7.8 Hz), 7.62 (1H, d, J=8.2 Hz), 7.30 (2H, d, J=9.0 Hz), 6.79 (1H, d, J=2.2 Hz), 6.70 (1H, dd, J=8.2, 2.2 Hz), 6.62 (2H, d, J=9.0 Hz), 4.62 (1H, t, J=5.2 Hz), 3.97 (2H, d, J=5.2 Hz), 3.42 (3H, s), 3.31 (4H, q, J=6.9 Hz), 2.90 (1H, d, J=15 Hz), 2.87 (1H, d, J=15 Hz), 2.63 (3H, s), 1.56 (3H, s), 1.12 (6H, t, J=6.9 Hz) ppm; ¹³C NMR (75 MHz, CDCl₃): δ 197.8, 170.8, 167.9, 154.3, 150.3, 148.1, 145.4, 144.9, 134.7, 130.1, 128.9, 125.6, 122.4, 122.3(3), 118.5, 113.1, 112.1(2), 108.1, 53.4, 51.4, 49.5, 48.2, 44.5, 43.6, 26.7, 25.9, 12.5(2) ppm.

1-24. Preparation of FHEt-1_{lyso}

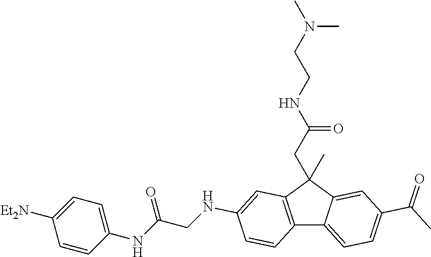

KOH (aq) (1 N, 5 mL, 5 mmol) was dropped to a solution of Compound 9 (0.26 g, 0.5 mmol) in EtOH (1 mL) while stirring at room temperature for 12 hours. The mixture was stirred at room temperature for 48 hours, and then dropped with HCl (aq) (1 N, 5 mL, 5 mmol). The formed precipitate was collected by filtration, washed with distilled water, dried in vacuum, and used in the next step without additional purification. HATU (0.13 g, 0.34 mmol) was added to a solution of the precipitate (0.13 g, 0.26 mmol), N,N-dimethylethylenediamine (0.034 g, 0.39 mmol), and (i-Pr)₂NH (0.090 mL, 0.52 mmol) in DMF (1 mL), and the mixture was stirred at room temperature for 12 hours. Distilled water was added, the product was extracted with EtOAc, and then the organic layer was washed with brine and dried with anhydrous Na₂SO₄ and the solvent was evaporated. The residue was purified through silica gel column chromatography using CH₂Cl₂/MeOH (1: 1) as a mobile phase to obtain FHEt-1_{lyso} in a yellow solid state (total yield 0.11 g, 40%).

¹H NMR (600 MHz, CDCl₃): δ 8.86 (1H, s), 8.55 (1H, s), 8.03 (1H, d, J=1.6 Hz), 7.92 (1H, dd, J=7.9 Hz, 1.6 Hz), 7.90 (1H, br s), 7.59 (1H, d, J=7.9 Hz), 7.57 (1H, d, J=8.3 Hz), 7.34 (2H, d, J=9.1 Hz), 6.84 (1H, d, J=2.2 Hz), 6.64 (1H, dd, J=8.3, 2.2 Hz), 6.62 (2H, d, J=9.1 Hz), 4.02 (1H, d, J=17 Hz), 3.98 (1H, d, J=17 Hz), 3.36-3.29 (1H, m), 3.31 (4H, q, J=7.0 Hz), 3.11-3.04 (1H, m), 2.94 (1H, d, J=14 Hz), 2.83 (1H, d, J=14 Hz), 2.68-2.63 (1H, m), 2.63 (3H, s), 2.57-2.52 (1H, m), 2.37 (6H, s), 1.53 (3H, s), 1.12 (6H, t, J=7.0 Hz) ppm; ¹³C NMR (75 MHz, CDCl₃): δ 198.0, 170.3, 168.4, 153.8, 150.5, 148.2, 145.2, 144.9, 134.5, 129.7, 128.6, 126.2, 122.7, 122.4(2), 122.2, 118.3, 112.2(2), 111.9, 108.8, 57.4, 48.9, 48.9, 45.4, 44.5(2), 43.0(2), 34.2, 27.2, 26.8, 12.4(2) ppm. HRMS (ESI) m/z [M+H]⁺ calcd for C₃₄H₄₃N₅O₃+: 570.3439, found: 570.3445.

### EXAMPLE 2. Experiment Method

2-1. Spectroscopy

Absorption and emission spectra of a two-photon probe (TP probe) were measured using an Agilent 8453 diode array UV-Vis spectrophotometer and a Hitachi fluorescence spectrophotometer F-7000 system using a previously reported method (Chem. Asian J. 2015, 10(10):2240-2249). Fluorescence quantum yield (Φ) was measured by a method used in another study (Anal. Chem. 2011, 83(4):1232-1242) using Coumarin 540A. Two-photon (TP) excitation was implemented using a mode-locked Ti:sapphire laser (Chameleon, 90 MHz, 200 fs; Coherent Inc.).

2-2. Water Solubility

The water solubility of BCa-1, BCa-2ₘᵢₜₒ, BCa−3°ₘₑₘ, and FHEt-1_{lyso} in an aqueous buffer (pH 7.4) was evaluated using a conventional fluorescence technique (Chem. Asian J. 2015, 10(10):2240-2249).

2-3. Measurement of TP Action Cross-Section (Φδ) and Effective TP Action Cross-Section (Φδ_{eff})

Φδ and Φδeff for the probes were evaluated using the fluorescence method as reported in other studies (Chem. Asian J. 2015, 10(10):2240-2249).

2-4. Measurement of Apparent Dissociation Constants

A dissociation constant (K_{d}) for binding the probe to Ca²⁺ was measured using a conventionally known method. Calcium calibration buffers (CBs; 10 mM 3-[N-morpholino]propanesulfonic acid [MOPS], 100 mM KCl, pH 7.2) containing Ca²⁺ at concentrations of 0 to 40 μM were prepared using a calcium calibration buffer kit (Biotium, CA, USA). A buffer containing 401.1M or more of Ca²⁺ was prepared by adding an appropriate amount of CaCl₂ to a MOPS buffer. Probes (BCa-3°ₘₑₘ at a concentration of 0.3 μM and BCa-1 and BCa-2ₘᵢₜₒ at concentrations of 0.1 μM) were added to calcium CB and fluorescence intensity was measured. Each K_{d} was calculated through a resultant titration curve. In addition, in order to evaluate a fluorescence titration of FHEt-1_{lyso}, the fluorescence intensity was measured by adding FHEt-1_{lyso} probe at a concentration of 0.6 μM in a universal buffer (UB; 0.1 M citric acid, 0.1 M KH₂PO₄, 0.1 M Na₂B₄O₇, 0.1 M tris[hydroxymethyl]aminomethane, 0.1 M KCl) at pH 4 to 11. The pKₐ value was calculated through a titration curve.

2-5. Preparation of Cells

HeLa cells were purchased from the Korean Cell Line Bank (Seoul, Korea). The cells were cultured by a method described in previous studies (Chem. Asian J. 2015, 10(10):2240-2249). Before two days of imaging, HeLa cells (5×10⁴/mL) were plated on a confocal glass bottom dish (#200350, SPL Life Sciences, Gyonggi-do, Korea). The cells were washed with an aqueous buffer (pH 7.4), a Hank's balanced salt solution (HBSS; LB003-02, WelGene Inc., Gyeongsangbuk-do, Korea), or a Ringer's solution (RB; 124 mM NaCl, 26 mM NaHCO₃, 10 mM d-glucose, 1.3 mM MgSO₄, 3 mM KCl, 1.25 mM NaH₂PO₄) unless otherwise specified. Thereafter, the probe (0.5 to 3 μM) and Pluronic® F-127 (0.03%, Sigma-Aldrich, St. Louis, MO, USA) were added to the cells in one of the three aqueous buffers (1.0 mL). The mixture was cultured (incubated) at 37° C. (BCa-1-AM and BCa-2ₘᵢₜₒ-AM) for 40 minutes or at room temperature (RT; BCa-3ₘₑₘ) for 10 minutes. The cells labeled with the probe were washed 3 times with each corresponding buffer before imaging.

2-6. Preparation of Tissues

Ex vivo brain slices were obtained from the hippocampus of 14-day-old Sprague-Dawley rats (Orient Bio Inc., Korea) according to a protocol approved by the institutional review board (IRB) of the Korea University. Fresh hippocampal slices (thickness: 400 μm) were prepared using a previously reported technique on an oscillating blade microtome (Chem. Asian J. 2015, 10(10):2240-2249).

2-7. Two-Photon Imaging (TP Imaging)

TP microscopy (TPM) images were obtained using spectral confocal and multiphoton microscopy (Leica TCS SP2; Leica). TPM images of probe-labeled HeLa cells were obtained using a Leica TCS SP2 using a conventional method (Chem. Asian J. 2015, 10(10):2240-2249). Time-dependent changes in TPEF intensity were monitored and measured at intervals of 1.6 to 2.0 seconds in the xyt mode upon excitation at 750 nm. To monitor changes in [Ca²⁺ ] in the plasma membrane, the cells were washed successively with RB containing 5, 0.1, and 0 mM of EGTA and then incubated with BCa-3ₘₑₘ (2 μM). The TPEF intensity was measured before and after adding CaCl₂ (2 mM) to RB and EGTA (2 mM) in distilled water.

Before and after adding histamine dihydrochloride (100 μM, Sigma-Aldrich) to HBSS, Ca²⁺ oscillation was evaluated by TPEF intensity monitoring in HeLa cells labeled with BCa-1-AM (2 μM).

Before and after adding carbonyl cyanide p-trifluoromethoxyphenylhydrazone (FCCP; 0.5 M, cat. #15218, Cayman, USA) to HBSS, changes in mitochondrial Ca²⁺ were analyzed by monitoring the TPEF intensity in HeLa cells labeled with BCa-2ₘᵢₜₒ-AM (2 μM).

To simultaneously monitor changes in lysosomal pH and cytoplasmic [Ca²⁺], HeLa cells were co-labeled with a mixture of BCa-1-AM (3 μM) and FHEt-1_{lyso} (1 μM). The cells were washed twice with RB, once with RB containing 5 mM EGTA, and twice with RB containing 0.1 mM EGTA. The TPEF intensity of the cells was measured before and after adding monensin (20 μM, Sigma-Aldrich) to EtOH.

For tissue imaging, brain slices were labeled with one of the following: BCa-1-AM (7 mM) and FHEt-1_{lyso} (3 mM) for 60 minutes at 37° C. for dual-color imaging, BCa-2ₘᵢₜₒ-AM (7 μM) for 60 minutes at 37° C. for mitochondrial imaging, or BCa-3ₘₑₘ (2 μM) for 10 minutes at RT for plasma membrane imaging. TPM images were obtained through the method described above except that six cross-sectional images were captured at 10 μm intervals at 90 to 140 μm depths from the tissue surface along a z-direction to minimize errors due to surface damage occurring during tissue preparation.

2-8. Cytotoxicity

The cytotoxicity of BCa-1-AM, BCa-2ₘᵢₜₒ-AM, BCa-3ₘₑₘ, and FHEt-1_{lyso} was measured using Cell Counting Kit-8 (Dojindo, Japan).

2-9. Photostability

Photostability was measured by monitoring a time-dependent decrease in TPEF intensity under imaging conditions for HeLa cells labeled with BCa-1-AM, BCa-2ₘᵢₜₒ-AM, BCa-3ₘₑₘ, or FHEt-1_{lyso}.

### EXAMPLE 3. Spectroscopic Characteristics and Fluorescence Titration of Two-Photon Probes

Table 1 showed results of analyzing the physical properties of the two-photon (TP) probes BCa-1-AM, BCa-2ₘᵢₜₒ-AM, BCa-3ₘₑₘ, and FHEt-1_{lyso} prepared in Example of the present disclosure.

TABLE 1

| | λₘₐₓ | | | | K_{d}^{g} or | | | |
|---|---|---|---|---|---|---|---|---|
| Probe | (λ_{fl})^{b} | 10⁻⁴ε^{c} | Φ^{d,e} | ²λₘₐₓ^{f} | pKₐ^{h} | FEFⁱ | Φδₘₐₓ^{j} | Φδ_{eff}^{k} |
| BCa-1 | 353 | 3.00 | 0.063^{d} | 470 | 0.18^{g} | 16 | 55 | 1400 |
| | (466) | | (0.99) | (461) | (nd) | (nd) | | |
| BCa-2ₘᵢₜₒ | 358 | 3.05 | 0.049^{d} | 470 | 2.7^{g} | 10 | 70 | 2000 |
| | (464) | | (0.50) | (462) | (nd) | (nd) | | |
| BCa-3°ₘₑₘ | 350 | 3.10 | 0.027^{d} | 470 | 100^{g} | 26 | 60 | 4800 |
| | (464) | | (0.72) | (427) | (95) | (25) | | |
| FHEt-1_{lyso} | 359 | 2.80 | 0.014^{e} | 562 | 6.5^{h} | 25 | 70 | 21000 |
| | (571) | | (0.33) | (550) | (6.6) | (24) | | |

^{a} λₘₐₓ of one-photon absorption spectra in nanometer unit.
^{b}λ_{fl} of one-photon absorption spectra in nanometer unit.
^{c}Molar extinction coefficient.
^{d,e}Fluorescence quantum yield (d) in the absence or presence of excess Ca²⁺ (in parentheses) and fluorescence quantum yields (e) at pH 11 and pH 4.3 (in parentheses).
^{f}λₘₐₓ of TPEF spectra in nanometer unit in aqueous buffer or Hela cells (in parentheses).
^{g,h}Dissociation constant (g) in micrometer unit and negative log (h) of acid dissociation constant.
ⁱFluorescence enhancement factor.
^{j}Maximum TP action cross-section at 10⁻⁵⁰ cm⁴s/photon (GM), error range: ±15%.
^{k}Efficient TP action cross-section in GM.

3-1. Spectroscopic Characteristics

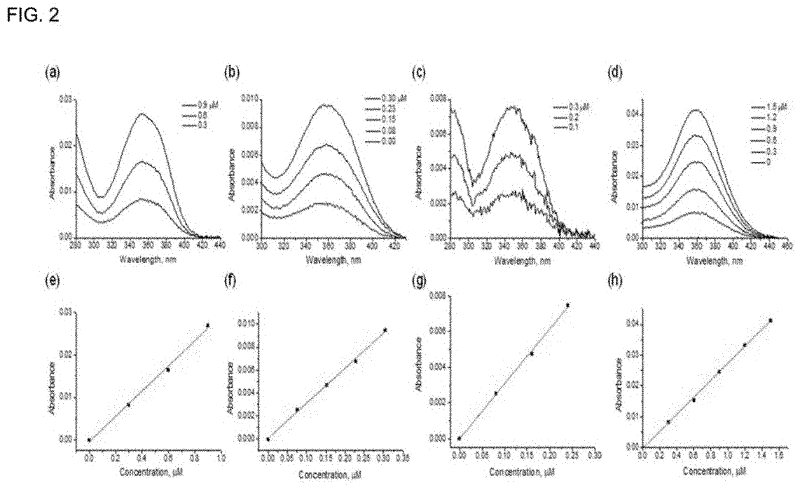

BCa-1, BCa-2 onto, and BCa-3°ₘₑₘ showed a molar extinction coefficient (c) of 30,000 to 31,000 cm-1M-1 and the maximum absorbance (λₘₐₓ) of 350 to 358 nm, as a result of a spectral study performed in phosphate buffered saline (pH 7.4; FIG. 2). All of the two-photon probe compounds emitted weak fluorescence at a maximum emission value (4) of 464 to 466 nm and fluorescence quantum yields (0) of 0.027 to 0.063 in CB (10 mM MOPS, 100 mM KCl, pH 7.2). FHEt-1_{lyso} showed a maximum absorbance (λₘₐₓ) at 359 nm with a molar extinction coefficient (c) of 28,000 cm-1M-1 in UB (FIG. 2 and Table 1), and the fluorescence quantum yield (0) was 0.014 and showed the maximum emission value (λ_{fl}) at 571 nm. The low fluorescence quantum yield (0) values observed with these probes may contribute to efficient photo-induced electron transfer (PeT) from the receptor to the fluorophore (Table 1).

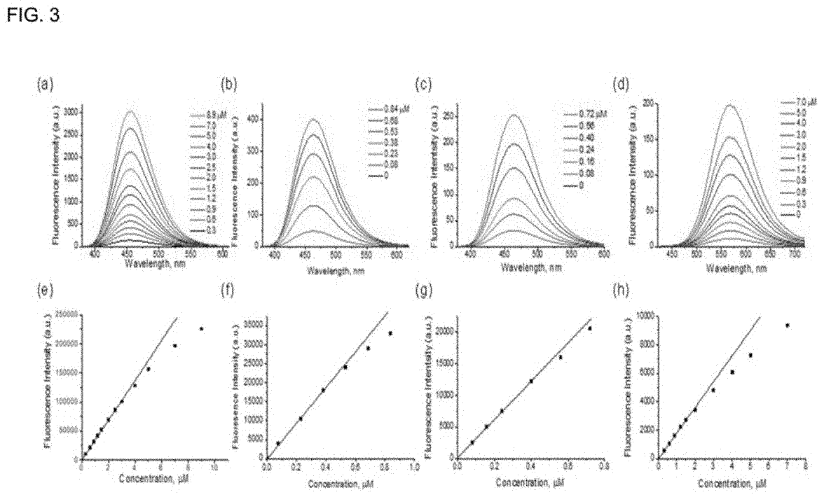

The water solubility measured by the absorption method for each probe compound was found to be 0.4 to 2.0 μM, which was sufficient to stain (color) cells (FIG. 3).

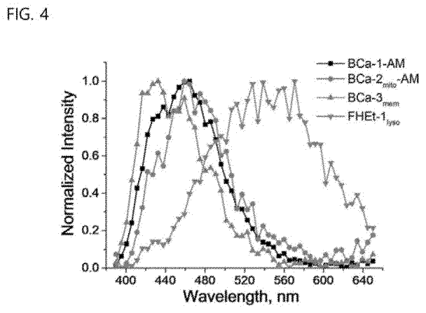

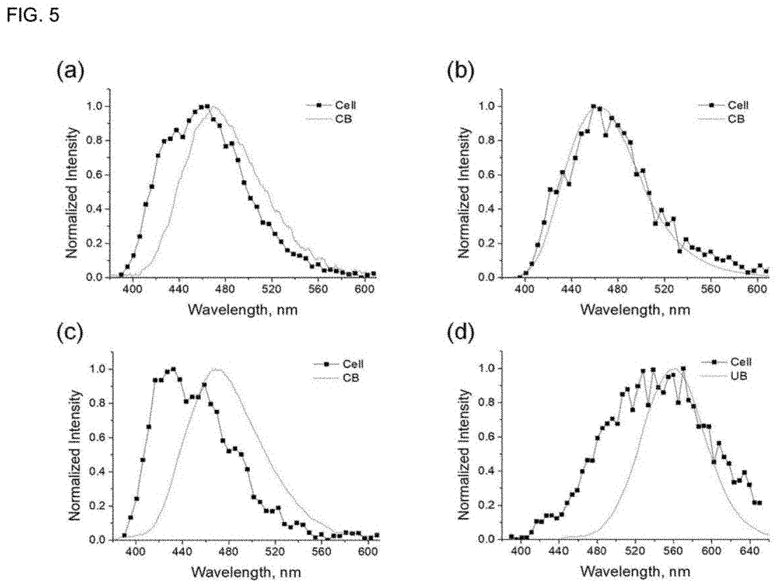

HeLa cells (cervical cancer cell line) labeled with BCa-3ₘₑₘ, BCa-1-AM, BCa-2ₘᵢₜₒ-AM, or FHEt-1_{lyso} emitted broad TPEF spectra based on 427, 461, 462, and 550 nm, respectively, when excited at 750 nm in a scanning lambda mode (FIG. 4). The TPEF maximum values of BCa-3., BCa-1-AM, BCa-2ₘᵢₜₒ-AM, and FHEt-1_{lyso} were blue-shifted by 43, 9, 8, and 12 nm from λ_{fl} measured in the aqueous buffer, respectively. These results indicate that the intracellular microenvironment is more hydrophobic than water and the microenvironment of the BCa-3ₘₑₘ probe is the most hydrophobic (FIG. 5).

3-2. Fluorescence Titration

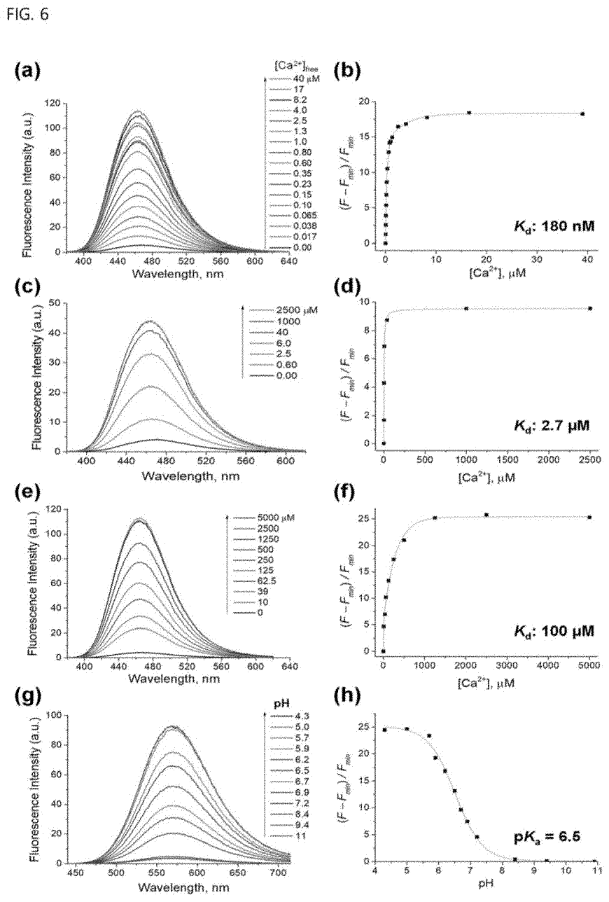

When Ca²⁺ was added little by little to BCa-1, BCa-2ₘᵢₜₒ, and BCa−3°ₘₑₘ in CB, the fluorescence intensity was increased rapidly (FIGS. 6 and S5). This can be expected to be achieved by efficiently blocking PeT from the receptor to the fluorophore when binding to Ca ions. The fluorescence enhancement factor [FEF=(Fₘₐₓ-Fₘᵢₙ)/Fₘᵢₙ] calculated for BCa-1, BCa-2ₘᵢₜₒ, and BCa−3°ₘₑₘ was at levels of 10 to 26 (FIG. 6 and Table 1). A titration curve showed an excellent correlation assuming a 1: 1 complexation between the probe and Ca²⁺ (FIG. 6).

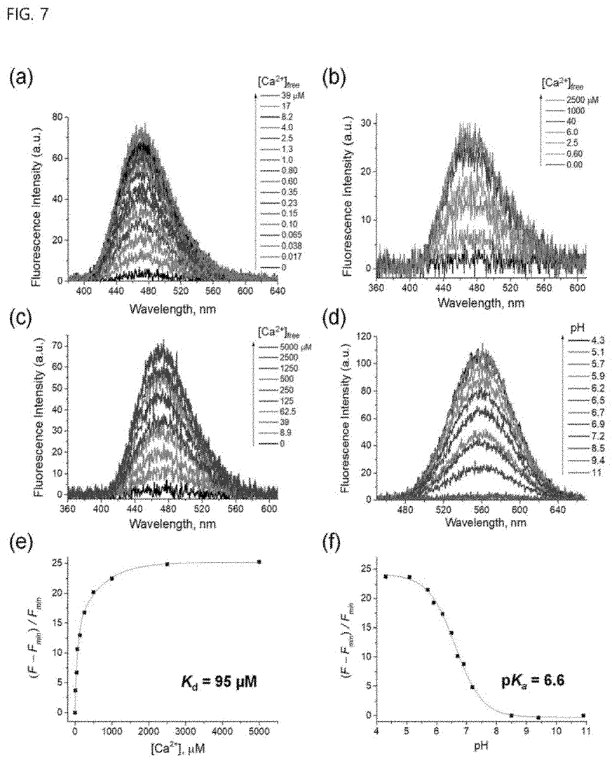

The dissociation constants (K_{d}) of BCa-1, BCa-2ₘᵢₜₒ, and BCa−3°ₘₑₘ calculated from the titration curve were 0.18, 2.7, and 100 μM, respectively. A similar value (K_{d}^{TP}=95 μM) for BCa-3°ₘₑₘ was determined in the TP process. The K_{d}^{TP} values for BCa-1 and BCa-2ₘᵢₜₒ were hardly calculated because the Fₘᵢₙ value of the TP process was too low to make accurate measurements (FIG. 7). K_{d} values were found to be within the range of intracellular concentrations of free Ca²⁺ in the corresponding organelles. These results indicate that the TP probe of the present disclosure may detect Ca ions bound to organelles in live tissue during TPM.

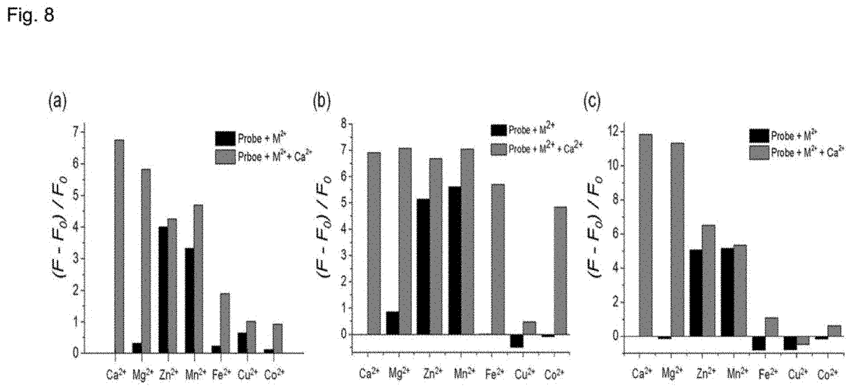

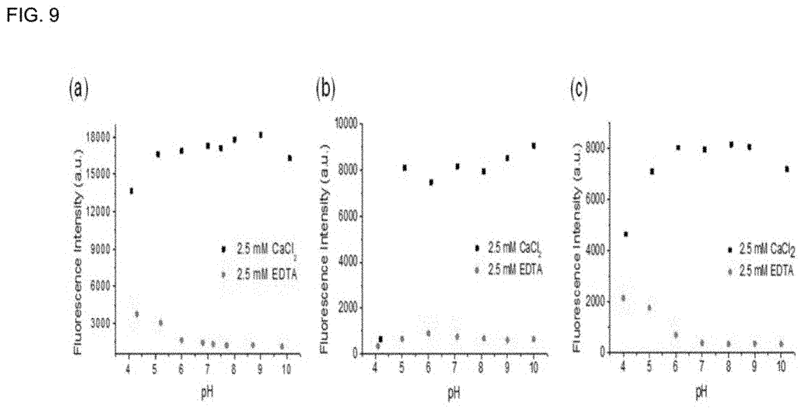

The BCa-1, BCa-2ₘᵢₜₒ, and BCa-3°ₘₑₘ probes of the present disclosure all showed weak responses to Mg²⁺ at 2 mM and Fe²⁺, Cu²⁺, and Co²⁺ at 100 μM, showed moderate responses to Zn²⁺ and Mn²⁺ at 100 μM, whereas showed high selectivity for Ca²⁺ (FIG. 8). In general, since the concentration of Zn′ in cells is in a nanomolar range and the concentration of Mn²⁺ may be negligible, it was confirmed that the two-photon fluorescent probe of the present disclosure may selectively detect Ca²⁺ without interference from other competing metal ions in cells. In addition, it was confirmed the two-photon fluorescent probe of the present disclosure showed low pH sensitivity at pH 4 to 10 (FIG. 9), thereby having pH-unresponsive characteristics, that is, pH independence in the pH range relevant to living organisms.

Meanwhile, the fluorescence titration of FHEt-1_{lyso} was performed in UB at pH 4 to 11. The pKₐ value of FHEt-1_{lyso} was 6.5, which was within the pH range of the cell (FIG. 6 and Table 1). Almost the same value (pKₐ^{TP}=6.6) was determined in the TP process (FIG. 7). Therefore, it was confirmed that FHEt-1_{lyso} may detect pH in live cells.

3-3. Brightness of Two-Photon Probe

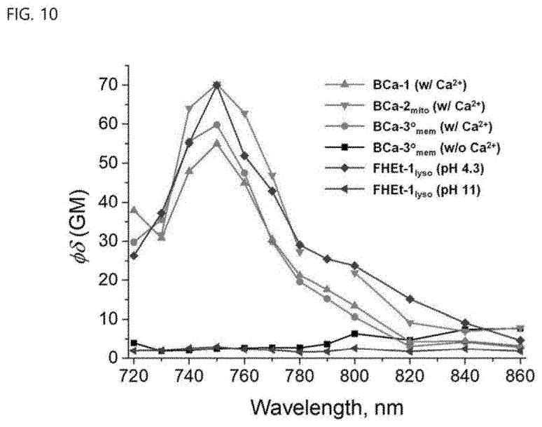

To evaluate the brightness of the TPM image, a TP action cross-section (Φδ) was measured by a conventionally known method. In the presence of excess Ca²⁺, the Φδ maximum values (Φδₘₐₓ) of BCa-1, BCa-2ₘᵢₜₒ, and BCa−3°ₘₑₘ were 55, 70, and 60 GM at 750 nm, respectively, whereas the Φδ maximum value of FHEt-1_{lyso} measured at pH 4.3 was 70 GM at 750 nm (FIG. 10). Due to the efficient PeT confirmed in Example, the Φδ values of BCa−3°ₘₑₘ and FHEt-1_{lyso} at pH 11 in the absence of Ca²⁺ were quite low.

In addition, the Φδ_{eff} value of the probe was determined in probe-labeled cells by comparing the TPEF intensity in MeOH with the intensity of 5.0 μM rhodamine 6 G. The Φδ_{eff} values of BCa-1, BCa-2ₘᵢₜₒ, BCa-3ₘₑₘ, and FHEt-1_{lyso} in HeLa cells were 1400, 2000, 4800, and 21000 GM at 750 nm, respectively, which were sufficient values to obtain bright TPM images. As a result of measuring the effective concentrations (c_{eff}) of BCa-1, BCa-2ₘᵢₜₒ, BCa-3ₘₑₘ, and FHEt-1_{lyso} using Equation of c_{eff}=Φδ_{eff}/Φδₘₐₓ, the bright spots were 25, 29, and 80, and 300 μM, respectively. These c_{eff} values were 17 to 600-fold higher than the probe concentrations measured in a staining medium, 1.5 μM for BCa-1, 1.0 μM for BCa-2ₘᵢₜₒ, and 0.5 μM for BCa-3ₘₑₘ and FHEt-1_{lyso}. These results indicate that the microenvironment of the probes is more favorable to the cells than the staining medium.

### EXAMPLE 4. Photostability and Cytotoxicity Analysis of Two-Photon Probes

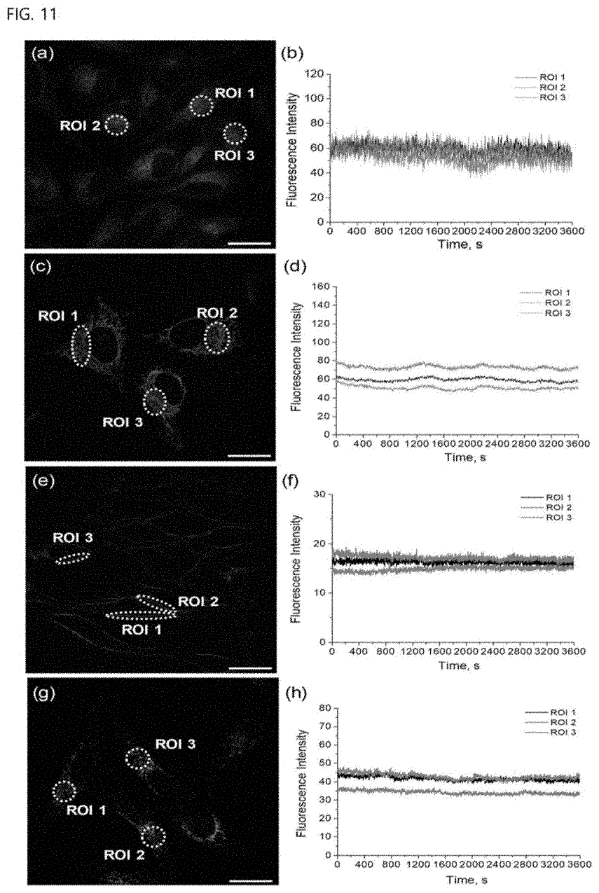

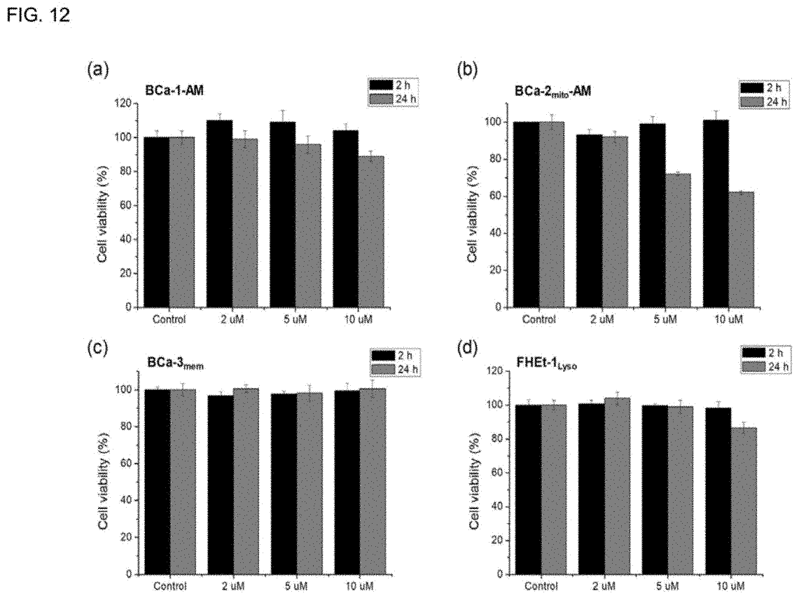

To evaluate the photostability of the probes, the probe was continuously irradiated with femtosecond pulses while monitoring the TPEF intensity of regions of interest (ROIs) in the TPM image of the probe-labeled cells (FIG. 11). The TPEF intensity values were maintained almost the same for 1 hour, and thus high photostability was shown. As a result of measurement with Cell Counting Kit-8, all the probes had low cytotoxicity (FIG. 12). From these results, it was confirmed that the probes had high photostability in relation to the TPM and may detect Ca²⁺ and H⁺ in organelles without cytotoxicity.

### EXAMPLE 5. Analysis of Detection Window of Two-Photon Probes

TPEF of HeLa cells labeled with BCa-3ₘₑₘ was measured at wavelengths of 380 to 660 nm. For colocalization experiments on HeLa cells co-labeled with BCa-2ₘᵢₜₒ-AM/MitoTracker Red′ (cat. #M22425, Invitrogen, Waltham, MA, USA), BLT-blue/FHEt-1_{lyso-}, and BCa-1-AM/FHEt-1_{lyso}, respectively, the detection window was measured using the TPEF spectra of the two probes to be compared. It was confirmed that the detection windows of the probes had well-separated emission bands and similar levels of emission intensities from the two probes to be compared.

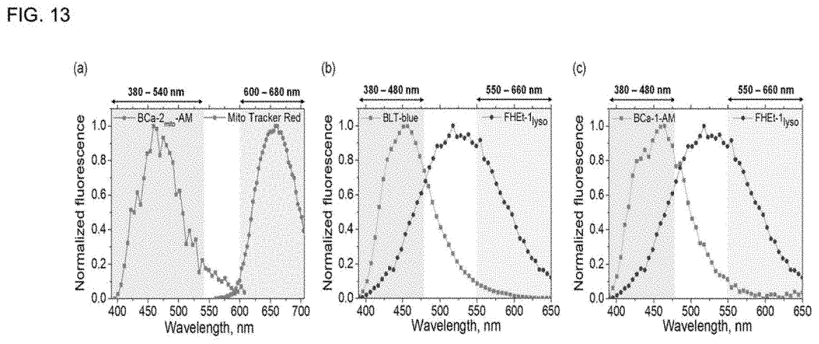

The detection windows for BCa-2ₘᵢₜₒ-AM, MitoTracker Red, BLT-blue, FHEt-1_{lyso}, and BCa-1-AM were 380 to 540, 600 to 680, 380 to 480, 550 to 660, and 380 to 480 nm, respectively (FIG. 13). Under the conditions, the TPEF ranges of BCa-2ₘᵢₜₒ-AM and MitoTracker Red did not overlap. In addition, FHEt-1_{lyso} fluorescence was responsible for 33% of BLT-blue fluorescence, BLT-blue fluorescence contributed to 3% of FHEt-1_{lyso} fluorescence, and FHEt-1_{lyso} fluorescence contributed to 33% of BCa-1-AM fluorescence, whereas BCa-1-AM fluorescence contributed to 3% of the FHEt-1_{lyso} fluorescence. Therefore, the TPEF values of BCa-2ₘᵢₜₒ-AM/MitoTracker Red′ (cat. #M22425, Invitrogen, Waltham, MA, USA), BLT-blue/FHEt-1_{lyso-}, and BCa-1-AM/FHEt-1_{lyso} probe pairs used for the colocalization experiments may be quantified with minimal interference with each other (FIG. 13).

### EXAMPLE 6. Evaluation of Organelles-Specific Detection Ability of Two-Photon Probes

6-1. Detection of Ca²⁺ in Plasma Membrane

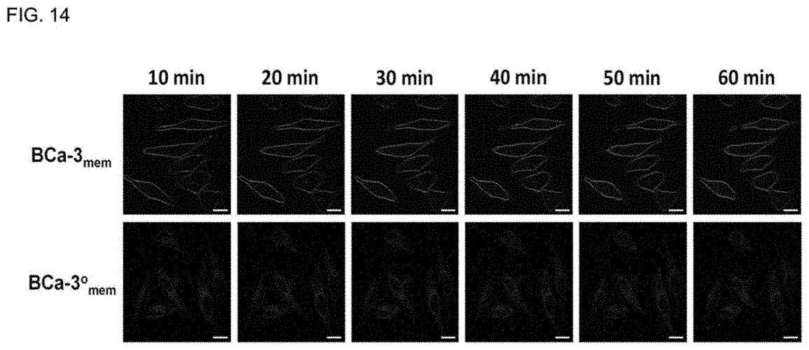

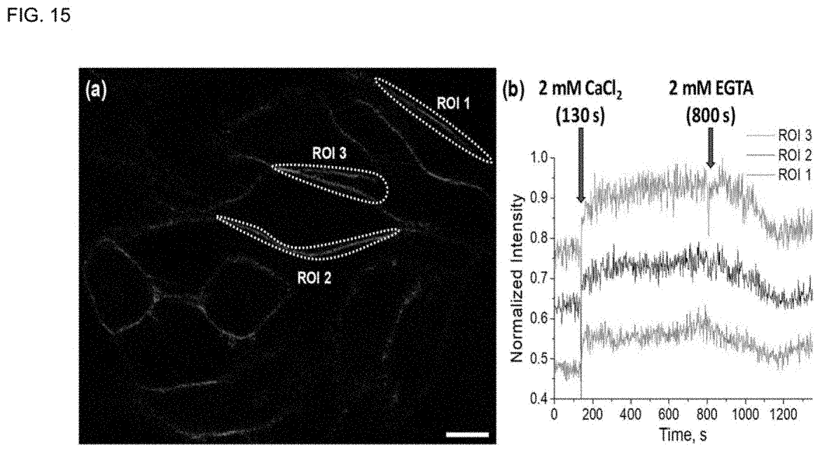

First, whether BCa-3ₘₑₘ may detect Ca²⁺ in the plasma membrane was evaluated. TPM images of HeLa cells labeled with BCa-3ₘₑₘ contained bright spots on the plasma membrane, and these signals persisted for at least 1 hour (FIG. 14). TPEF intensity increased upon addition of Ca²⁺ (2 mM) and decreased during treatment with EGTA (2 mM), a chelating agent for Ca²⁺ (FIG. 15). Accordingly, it was confirmed that BCa-3ₘₑₘ may target the plasma membrane to detect Ca²⁺ in the plasma membrane.

Meanwhile, in contrast to the results of BCa-3ₘₑₘ, the images were much darker and blurry when the cells were labeled with BCa−3°ₘₑₘ (FIG. 14). Through these results, it was confirmed that it was important to introducing a hydrocarbon tail with an optimal length in order to design a TP probe for membrane-bound targets.

6-2. Detection of Ca²⁺ in Cytoplasm

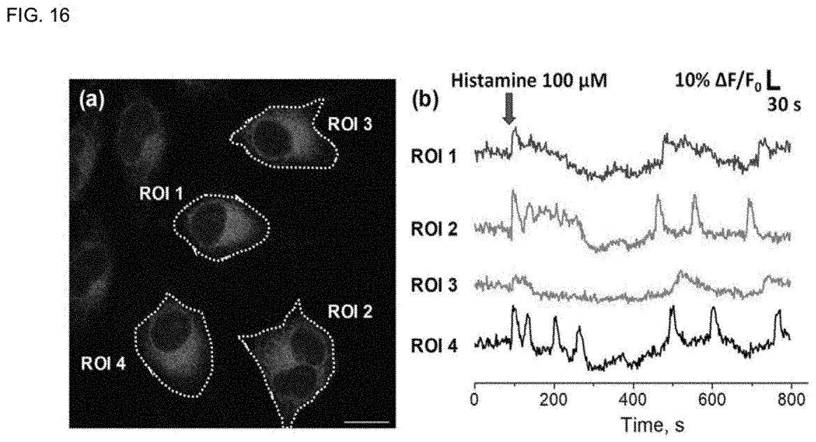

Whether BCa-1 may detect Ca²⁺ in the cytoplasm was evaluated. TPM images of HeLa cells labeled with BCa-1-AM showed bright spots in the cytoplasm. When adding histamine, a reagent that induces Ca oscillation, the TPEF intensity increased and then decreased. The increase and decrease of the TPEF intensity were repeated for 800 seconds, which was consistent with the results of previous histamine-induced Ca oscillation studies. Accordingly, it was confirmed that BCa-1 may target the cytoplasm and detect Ca²⁺ in the cytoplasm (FIG. 16).

6-3. Detection of Ca²⁺ in Mitochondria

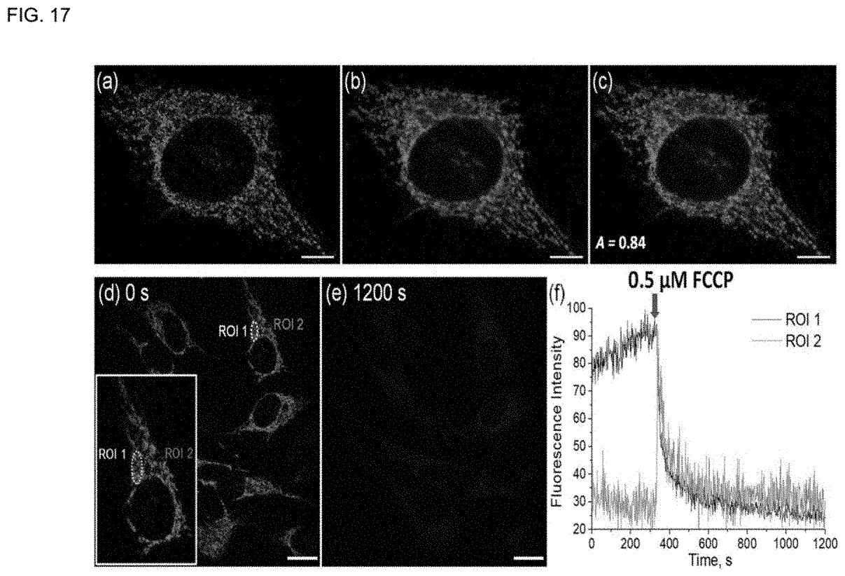

Whether BCa-2ₘᵢₜₒ-AM may detect Ca²⁺ in the mitochondria was evaluated. TPM images of HeLa cells labeled with BCa-2ₘᵢₜₒ-AM showed well-colocalized bright spots with MitoTracker Red having a Pearson's colocalization coefficient (A) of 0.84 (FIGS. 17A to 17C).

When the cells were treated with FCCP, a reagent that depolarizes the mitochondrial membrane, the TPEF intensity rapidly decreased in the mitochondria (FIG. 17F, black curve), and then the TPEF intensity increased temporarily in the cytoplasm and then gradually decreased to a baseline (FIG. 17F, red curve). These results were consistent with the fact that FCCP induced the release of Ca²⁺ from the mitochondria first to the cytoplasm and then to the extracellular space (FIGS. 17D to 17F). Therefore, it was confirmed that BCa-2ₘᵢₜₒ had the ability to detect Ca²⁺ in the mitochondria by targeting the mitochondria.

6-4. Detection of H⁺ in lysosome

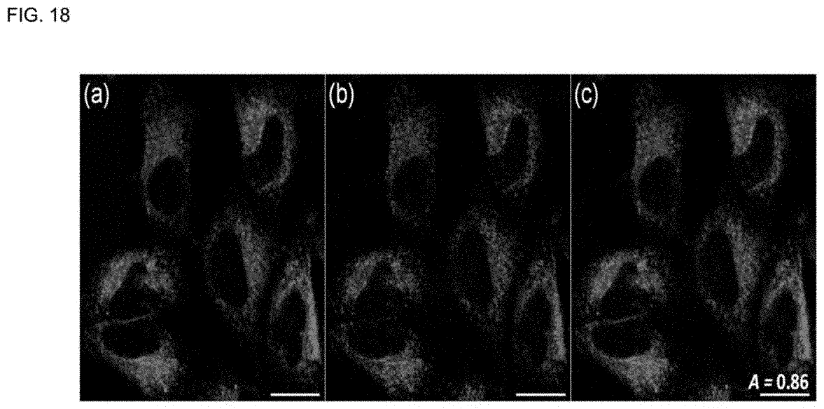

Whether FHEt-1_{lyso} may detect H⁺ in the lysosome was evaluated. TPM images of HeLa cells labeled with FHEt-1_{lyso} showed well-colocalized bright spots with BLT-blue having a Pearson's colocalization coefficient (A) of 0.86 as a TP probe for the lysosome (FIG. 18). Therefore, it was confirmed that FHEt-1_{lyso} had the ability to detect H⁺ in the lysosome by targeting the lysosome.

6-5. Dual-Color Imaging

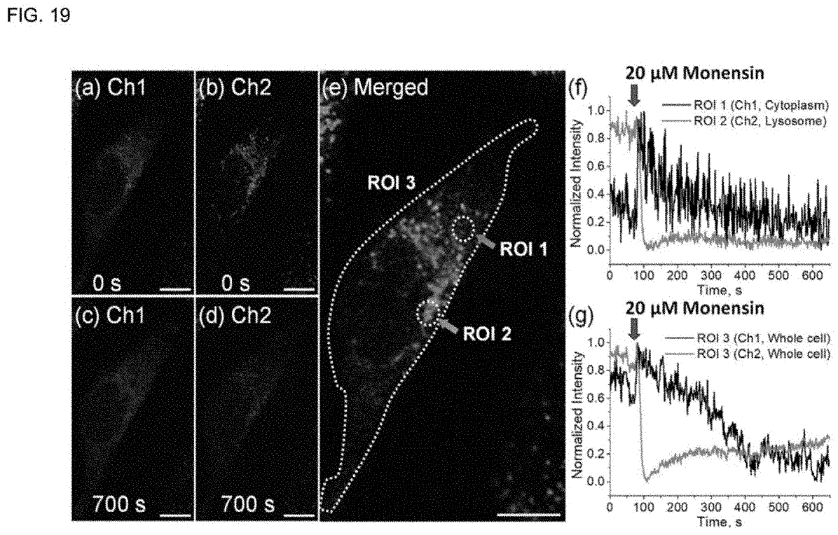

Whether BCa-1 and FHEt-1_{lyso} could be used to simultaneously monitor changes in cytoplasmic [Ca²⁺ ] and lysosomal pH during dual-color TPM was evaluated. TPM images of HeLa cells co-labeled with BCa-1-AM and FHEt-1_{lyso} showed Ca²⁺ in the cytoplasm (green pixels) and H⁺ in the lysosome (red pixels, FIGS. 19A to 19D). As the cytoplasmic Ca concentration and lysosomal pH increased when monensin was added to the cells, the TPEF intensity rapidly decreased in the lysosome (red curve) and simultaneously increased in the cytoplasm (black curve) (FIGS. 19F and 19G). These results were consistent with the increases in cytoplasmic Ca²⁺ concentration and lysosomal pH induced by monensin. Accordingly, it was confirmed that BCa-1 and FHEt-1_{lyso} could be used to simultaneously monitor changes in cytoplasmic [Ca²⁺] and lysosomal pH in live cells during dual-color TPM.

### EXAMPLE 7. Evaluation of Usefulness as TP Probe in Live Tissue

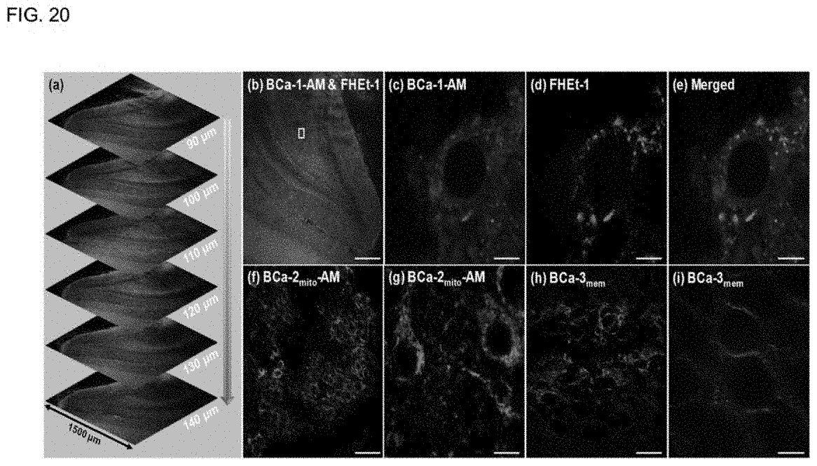

The BCa-1-AM and FHEt-1_{lyso} probes of the present disclosure were evaluated for usefulness as TP probes for tissue imaging. Hippocampal tissue slices from 14-day-old Sprague-Dawley rats were incubated with 7 M BCa-1-AM and 3 M FHEt-1_{lyso} at 37° C. for 60 minutes. Six TPM images were obtained at depths of 90 to 140 μm to visualize the distribution of cytoplasmic Ca²⁺ and lysosomal H⁺ ions at different depths on pyramidal neuron layers in CA1 and dentate gyrus regions (FIGS. 20A and 20B). Cross-sectional images were similar to each other and showed similar distributions of cytoplasmic Ca²⁺ and lysosomal H⁺ at different depths. In addition, images obtained at 380 to 480 nm (BCa-1-AM) and 550 to 660 nm (FHEt-1_{lyso}) at high magnification clearly showed the distribution of two ions at a depth of 100 μm (FIGS. 20C to 20E). As expected from being located in different organelles, the signals did not overlap between the two images (FIG. 20E). Thus, it was confirmed that BCa-1-AM and FHEt-1_{lyso} may simultaneously detect cytoplasmic Ca²⁺ and lysosomal H⁺ in live tissue in the context of a dual-color TPM.

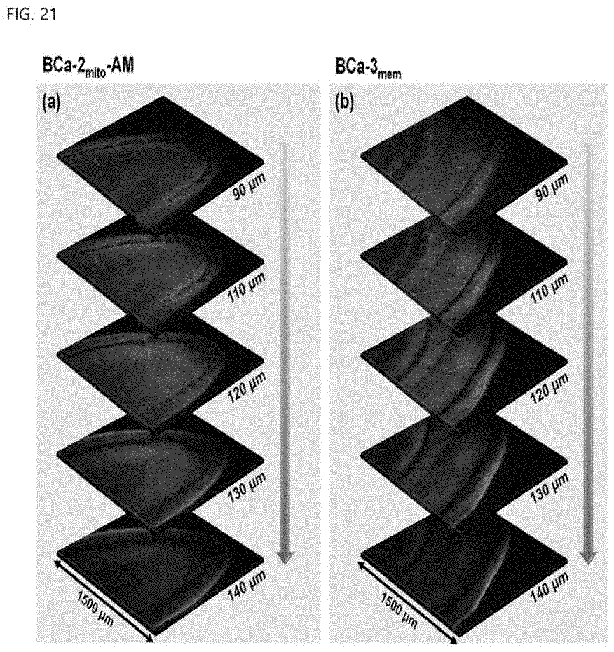

In addition, cross-sectional TPM images (depths of 90 to 140 μm) of hippocampal slices labeled with BCa-2ₘᵢₜₒ-AM and BCa-3ₘₑₘ were obtained (FIG. 21). Similarly to the results, these probes also showed a similar distribution of Ca ions in the mitochondria and plasma membrane in an xy plane over the entire analysis depth. In particular, images obtained at high magnification clarified the distribution of Ca ions in the mitochondria and plasma membrane at a depth of 100 μm (FIGS. 20F to 201).

Therefore, it was confirmed that BCa-2ₘᵢₜₒ-AM and BCa-3ₘₑₘ could detect Ca ions in the mitochondria and plasma membrane in live tissues during TPM, respectively.

### CONCLUSION

The present inventors have prepared blue emission TP probes (BCa-1-AM, BCa-2ₘᵢₜₒ-AM, BCa-3ₘₑₘ) for Ca²⁺ and a green emission TP probe (FHEt-1_{lyso}) for H⁺ capable of organelles-specific detection in the cytoplasm, mitochondria, plasma membrane, and the lysosome, respectively. These probes showed significant TP cross-section, high selectivity and sensitivity for Ca²⁺ to be detected, high photostability, low pH dependence, and negligible cytotoxicity. BCa-1-AM, BCa-2ₘᵢₜₒ-AM, and BCa-3ₘₑₘ may selectively detect Ca²⁺ in real time in the cytoplasm, mitochondria, and plasma membrane of live cells and tissues, respectively, and FHEt-1_{lyso} may detect lysosomal H⁺ in live cells and tissues. In addition, BCa-1-AM and FHEt-1_{lyso} may simultaneously detect cytoplasmic Ca²⁺ and lysosomal H⁺ in live cells and tissues through dual-color TPM images. Through these results, it was confirmed that the organelles-specific blue emission TP probe derived from 6-(benzoxazol-2-yl)-2-naphthalylamine of the present disclosure was useful as a TP Ca²⁺ probe capable of detecting Ca ions in the cytoplasm, mitochondria, and plasma membrane of live cells and tissues. In addition, the probe of the present disclosure can be used to examine crosstalk between metal ions through multicolor TPM imaging. Accordingly, the probe of the present disclosure may be useful for biomedical research including physiological research on living biological tissues.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A two-photon fluorescent probe compound represented by any one of Chemical Formulas 1 to 3 below: 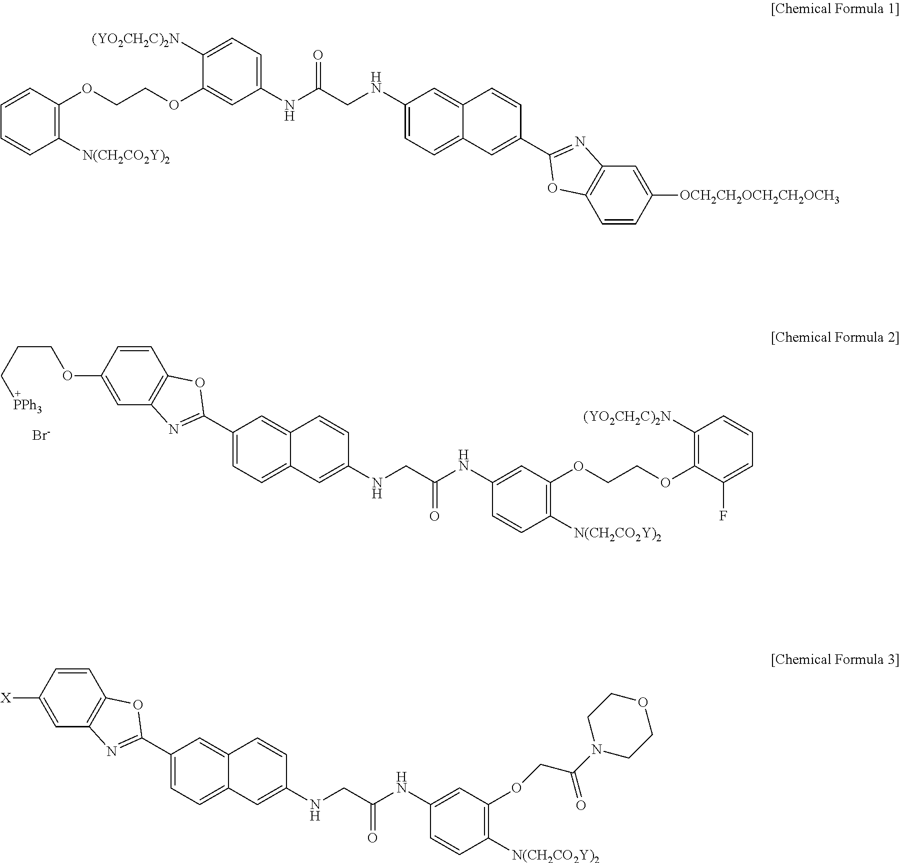
wherein, in Chemical Formulas 1 to 3, Y is each independently K, Na, CH₃, or CH₂OC(O)CH₃, and
in Chemical Formula 3, X is H, OH, or O(CH₂)₅CH₃.

2. The two-photon fluorescent probe compound of claim 1, wherein the two-photon fluorescent probe compound is for selectively imaging calcium ions (Ca²⁺) in a cytoplasm, mitochondria, or plasma membrane.

3. A two-photon fluorescent probe composition for detecting calcium ions in live cells or tissues comprising at least one compound selected from the two-photon fluorescent probe compounds according to claim 1.

4. A method for imaging calcium ions comprising steps of:
injecting at least one compound selected from the two-photon fluorescent probe compounds according to claim 1 into cells or tissues isolated from a living body;
irradiating an excitation source to the cells or tissues isolated from the living body; and
observing fluorescence generated from the two-photon fluorescent probe compound by two-photon microscopy.

5. A method for preparing the two-photon fluorescent probe compound represented by any one of Chemical Formulas 1 to 3 of claim 1, comprising reacting a compound represented by Chemical Formula 5 below with a compound represented by Chemical Formula 6 or 7 below: 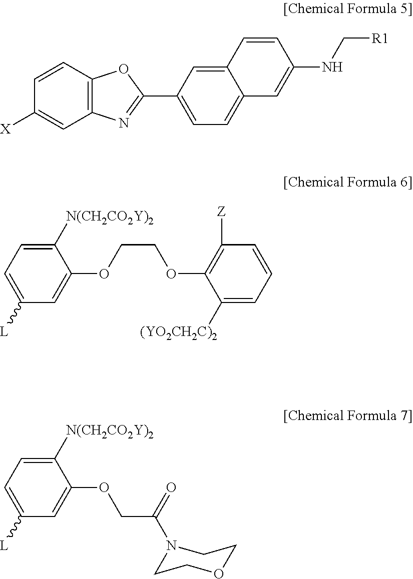
wherein, in Chemical Formula 5, 6, or 7, Y is each independently K, Na, CH₃, or CH₂OC(O)CH₃,
X is H, OH, O(CH₂CH₂O)₂CH₃, O(CH₂)₃Br, O(CH₂)₃PPh₃₊₁₃f, or O(CH₂)₅CH₃,
R1 is CO₂C(CH₃)₃ or CO₂H,
Z is H or F, and
L is H or NH₂.

6. The method for preparing the two-photon fluorescent probe compound represented by any one of Chemical Formulas 1 to 3 of claim 5, wherein in Chemical Formula 1, Y is K or CH₂OC(O)CH₃, in Chemical Formula 5, X is OH, R1 is CO₂H, and in Chemical Formula 6, Y is CH₃, Z is H, and L is NH₂.

7. The method for preparing the two-photon fluorescent probe compound represented by any one of Chemical Formulas 1 to 3 of claim 5, wherein in Chemical Formula 2, Y is K or CH₂OC(O)CH₃, in Chemical Formula 5, X is OH and R1 is CO₂H, and in Chemical Formula 6, Y is CH₃, Z is F, and L is NH₂.

8. The method for preparing the two-photon fluorescent probe compound represented by any one of Chemical Formulas 1 to 3 of claim 5, wherein in Chemical Formula 3, Y is K or CH₃ and X is O(CH₂)₅CH₃ or H, in Chemical Formula 5, X is H or OH and R1 is CO₂H, and in Chemical Formula 7, Y is CH₃ and L is NH₂.

9. A two-photon fluorescent probe compound represented by Chemical Formula 4 below: 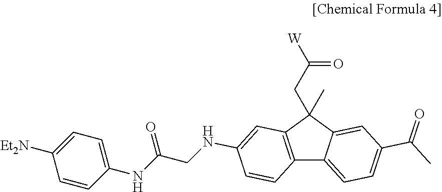
wherein in Chemical Formula 4, W is NH(CH₂)₂N(CH₃)₂ or OCH₃.

10. The two-photon fluorescent probe compound of claim 9, wherein the two-photon fluorescent probe compound is for selectively imaging hydrogen ions in a lysosome.

11. A two-photon fluorescent probe composition for detecting hydrogen ions in live cells or tissues, comprising the two-photon fluorescent probe compound according to claim 9.

12. A method for imaging hydrogen ions comprising:
injecting the two-photon fluorescent probe compound according to claim 9 into cells or tissues isolated from a living body;
irradiating an excitation source to the cells or tissues isolated from the living body; and
observing fluorescence generated from the two-photon fluorescent probe compound by two-photon microscopy.

13. A method for preparing the two-photon fluorescent probe compound represented by Chemical Formula 4 of claim 9, comprising reacting a compound represented by Chemical Formula 8 below with a compound represented by Chemical Formula 9 below: 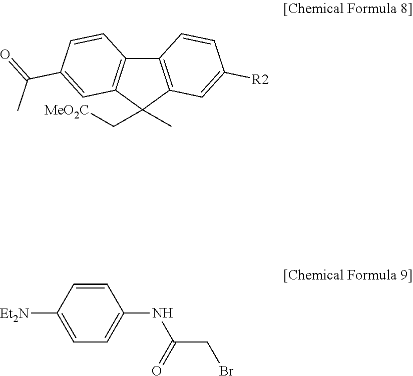
wherein in Chemical Formula 8, R2 is NO₂ or NH₂.

14. A two-photon fluorescent probe composition for simultaneously detecting calcium ions and hydrogen ions comprising at least one compound selected from the two-photon fluorescent probe compound represented by any one of Chemical Formulas 1 to 3 of claim 1 and a compound represented by Chemical Formula 4 below 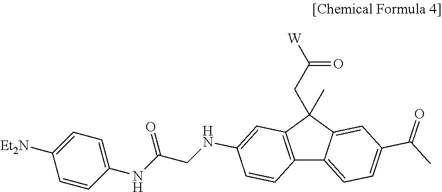
wherein in Chemical Formula 4, W is NH(CH₂)₂N(CH₃)₂ or OCH₃.

15. The two-photon fluorescent probe composition for simultaneously detecting calcium ions and hydrogen ions of claim 14, wherein the two-photon fluorescent probe compound represented by any one of Chemical Formulas 1 to 3 targets a cytoplasm, mitochondria, or plasma membrane to selectively image the calcium ions in the cytoplasm, mitochondria, or plasma membrane, and the compound represented by Chemical Formula 4 targets a lysosome to selectively image the hydrogen ions in the lysosome.

16. A method for simultaneously imaging calcium ions and hydrogen ions comprising:
injecting at least one compound selected from the two-photon fluorescent probe compound represented by any one of Chemical Formulas 1 to 3 of claim 1 and a two-photon fluorescent probe compound represented by Chemical Formula 4 below into cells or tissues isolated from a living body 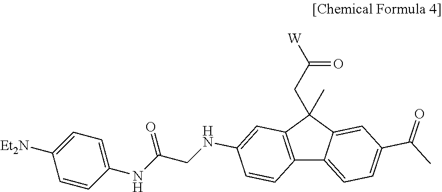
wherein in Chemical Formula 4, W is NH(CH₂)₂N(CH₃)₂ or OCH₃;
irradiating an excitation source to the cells or tissues isolated from the living body; and
observing fluorescence generated from the two two-photon fluorescent probe compounds by two-photon microscopy.

17. The method for simultaneously imaging calcium ions and hydrogen ions of claim 16, wherein the two-photon fluorescent probe compound represented by any one of Chemical Formulas 1 to 3 targets a cytoplasm, mitochondria, or plasma membrane to selectively image the calcium ions in the cytoplasm, mitochondria, or plasma membrane, and the compound of Chemical Formula 4 targets a lysosome to selectively image the hydrogen ions in the lysosome.

18. The method for simultaneously imaging calcium ions and hydrogen ions of claim 16, wherein the imaging is simultaneously imaging changes in distribution of the calcium ions in the cytoplasm, mitochondria, or plasma membrane and acidity (pH) of a lysosome.
